(19) United States
(12) Patent Application Publication
(10) Pub. No.: US 2006/0099712 A1
(43) Pub. Date: May 11, 2006

(54) Correlation of anti-cancer activity of dyes with redox potentials

(75) Inventors: Paul B. Gilman JR., Penfield, NY (US); Richard L. Parton, Webster, NY (US); Jerome R. Lenhard, Fairport, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/983,369

(22) Filed: Nov. 8, 2004

(51) Int. Cl. G01N 33/00

(52) U.S. Cl. 436/64

(57) ABSTRACT

The present invention relates to a method for selecting pharmacological compounds for selective inhibition of cancer cells comprising identifying a compound, determining the reduction potential (E_{R}) of the compound, and selecting the compound which has a reduction potential from −1.1 to −0.8 volts. The invention also relates to a pharmacological compound comprising at least one cyanine dye or merocyanine dye, wherein the dye has at least one cationic substituent, wherein the dye has a reduction potential of from—1.1 to 0.8 volts, and wherein the pharmacological compound demonstrates selective inhibition of cancer cells.

### FIELD OF THE INVENTION

The present invention relates to the use of electrochemical reduction potential as a factor in determining compounds, particularly cyanine and merocyanine dyes, with anti-cancer activity. Furthermore, the present invention relates to cyanine and merocyanine dyes with anti-cancer activity, which have reduced toxicity in biological systems.

### BACKGROUND OF THE INVENTION

It is well recognized that cancer is a scourge of the modern world, particularly of the developed nations. This is particularly devastating in view of the pain and incapacity which proceeds actual death by cancer. It is not surprising, therefore, that much attention is being given to discovering anti-cancer agents. The need for effective anti-cancer agents is so well known that whenever it is rumored that one has been found the press and the public clamor for information.

Generally, anti-cancer agents are grouped into antibiotic and immunological types. Since both of these types of anti-cancer agents do not distinguish between cancer and normal cells, they are strongly toxic to normal cells and unpromising in the conquest of malignant tumors. These toxic effects typically produce undesirable side effects, such as alopecia (hair loss), emisis (nausea), nephrotoxicity, cardiotoxicity, to name a few. Another problem has been the relative lack of success when using even the most popular drugs. Yet another disadvantage relates to the difficulty with respect to the large-scale preparation of anti-cancer agents, which requires high-purity and pyrogen-free form.

Yet, so severe is the problem of cancer that people take such drugs and suffer the side effects in the hope that the cancer will be alleviated before the side effects become unbearable. There is currently available some selectivity in toxicity, that is, the ability of the chemotherapeutic agent to selectively kill carcinoma cells instead of healthy cells. However, for most such conventional chemotherapeutic agents, that selectivity does not exceed 2:1 or 3:1 as defined, for example, by the inhibitory concentration at which 50% of the cells of the tested culture are killed, IC₅₀ values, in in vitro studies of human carcinomas. Such conventional selectivity of 2:1 or 3:1 is not adequate because undesirable side effects still plague the patient. Selectivity values defined by the IC₅₀ ratios of at least 5:1 are needed before the selectivity becomes significant enough to predict reduced undesirable side effects.

Methine dyes (also referred to as methylidyne dyes) comprise a methine chain (i.e., a chain of carbon atoms with alternating double and single bonds) terminated at each end with a heteroatom. The terminal heteroatoms are typically nitrogen or oxygen atoms in various combinations, and generally they are contained in or attached to an unsaturated cyclic nucleus. For example, cyanine dyes comprise a methine chain terminated at each end with a nitrogen heteroatom, wherein the nitrogen heteroatoms are part of a heterocyclic ring. In cyanine dyes a positive charge is delocalized between the two nitrogens.

A large number of structural variations of methine dyes have been explored including various heterocycles and substituents on the methine chain to affect dye hue and to obtain desirable physical and photographic properties. The measured electrochemical oxidation and electrochemical reduction potentials of cyanine dyes have been used to correlate the lowest and highest occupied molecular orbitals of the dye with its ability to sensitize or desensitize a photographic emulsion. (See P. B. Gilman, Pure and Appl. Chem., 49, 357 (1977); R. L. Large, in Photographic Sensitivity, R. Cox, Ed., Academic Press, New York, 1973, p 241; T. Tani et al., Journal of the Electrochemical Society, 138, 1411 (1991)). U.S. Pat. No. 4,232,121 discloses a method for identifying methine dyes which inhibit cell growth by (1) determining the reduction potential of the dye, (2) determining the ability of the dye to adsorb to the cells and (3) selecting those dyes which have a reduction potential more negative than about —0.8 volt and which are adsorbed to the cells. Typical methine dyes are cyanine dyes, merocyanine dyes and oxonol dyes. Reduction potentials for numerous methine dyes are available in the literature, as are suitable techniques for measuring them.

These dyes have found wide use in photography and related arts where they are employed, inter alia, as spectral sensitizers. The dyes are used to extend the spectral response of silver halide and other photosensitive materials to regions of the spectrum where they do not have inherent or native sensitivity. Specific methine dyes have been used as anthelmintic and antifilarial agents and the efficacy of a number of methine dyes as bactericides has been investigated.

Many studies have been made which correlate the electrochemical reduction and oxidation potentials of cyanine dye with the electron transfer photographic properties in silver halide systems. Some published studies are:

(1) Antifoggant Behavior by Electron Trapping Dyes: P. B. Gilman, T. D. Koszelak, A. A. Adin and R. G. Willis, U.S. Pat. No. 4,933,273 (1990);

(2) Anomalously Efficient Spectral Sensitization: A. A. Muenter, P. B. Gilman, J. Lenhard and T. L. Penner, The International East-West Symposium on The Factors Influencing Photographic Sensitivity Pre print Book, Page C-21 (1984);

(3) Biological Influence of Dyes and Anti-cancer Effects: S. Zigman and P. B. Gilman, Science, 208:188 (1980); W. Humphlett and L. B. Chen, Europ. Pat. 2862,252 (1988); J. Medicinal Chemistry 21:11(1978); P. B. Gilman, R T. Belly, T. D. Koszelak and S. Zigman, U.S. Pat. NO. 4,232,121(1980);

(4) Chemical Sensitization: I. H. Leubner, Photog. Sci. Eng., 20: (1976);

(5) Photohole Formation: R. W. Beriman and P. B. Gilman, Photogr. Sci. and Eng. 17: 235 (1974);

(6) Effect of Dyes on Latent Imagine Keeping: T. Tani, J. Soc. Photog. Sci. and Tech. Japan: 43: 27 (1980); T. Tani, J. Soc. Photog. Sci. and Tech. Japan 43: 335 (1980);

(7) Use of Dyes To Calibrate Energy Levels of Chemical Sensitizers and Dopants: P. B. Gilman, T. L. Penner, T. D. Koszelak and S. K Mroczek, “Progress in Basic Principles of Imaging Systems”, Proceedings of the International Congress of Photographic Science, Cologne, 1986 Ed. F. Granzer and E. Moisar, P 228 (1986);

(8) The Limiting Factors for Spectral Sensitization and Their Cures: P. B. Gilman, J. Signal A M, 1: 5 (1976);

(9) Speral Sensitization of Core-Shell Emulsions: F. J. Evans and P. B. Gilman, Photogr. Sci. and Eng. 19: 333 (1975);

(10) Spectral Sensitization of Sub-Conduction Band Events: P. B. Gilman and T. L. Penner, Photogr. Sci. and Eng. 28: 238 (1984);

(11) Self Supersensitization of Dye Aggregates: P. B. Gilman and T. D. Koszelak, J. Photogr. Sci. 21: 53 (1974);

(11) Spectral Sensitizing Thresholds for Different Silver Halides, P. B. Gilman, Photogr. Sci and Eng. 18.475 (1974);

(12) Mechanisms of Supersensitization: P. B. Gilma, Photogr. Sci. and Eng. 18:418 (1974).

It has been found that one of the most useful tools to understand and control electron transfer reactions in silver halide photographic systems has been to use a series of dyes which vary systematically in either electrochemical reduction or oxidation potential.

By using such a series of dyes, nearly all of the important electron transfer reactions occurring in the silver halide photographic process have been identified and controlled. Because a series of dyes varying systematically in the electrochemical reduction or oxidation potential has been so successful for the identification and control of electron transfer reactions in silver halide, it was hypothesized that a similar series of dyes varying systematically in either electrochemical reduction or oxidation potential might also be useful for the identification and control of electron transfer processes in biological systems. In 1976, through advertisements published in Science (April 30) and Scientific American (May) and included herein as FIG. 1, a set of 18 cyanine dyes varying in electrochemical redox properties was made available by Kodak to the scientific community.

Dr. Lan Bo Chen at the Dana-Farber Cancer Institute (which is associated with Harvard University) had investigated pyrilium dyes and Rhodamine 123 (Rh123), which is commercially available from Kodak. Dyes of this type were found to have accumulated in mitochondria of living cells. (See M. J. Weiss, L. B. Chen, Kodak Laboratory Chemicals Bulletin, 55, 1, (1984).) Increased accumulation and retention by carcinoma cells for Rh123 were also identified, as well as Rh123 selective toxicity to carcinoma cells in vitro. Subsequently, a large number of pyrylium and thiapyrylium dyes which had been prepared over the years in the Research Laboratories have been screened by Dr. Chen. A number of these dyes, especially the thiapyryliums, show excellent anti-cancer activity. However, some general disadvantages of the thiapyryliums are poor water solubility, which would make them difficult to administer, and accumulation in the kidneys, which could have toxic effects.

Certain types of cyanine dyes have been disclosed as having anti-cancer activity, for example, in JP80/69513, JP80/100318, JP89/52325, U.S. Pat. No. 5,618,831, U.S. Pat. No. 5,491,151, U.S. Pat. No. 5,670,530, U.S. Pat. No. 5,861,424, U.S. Pat. No. 5,360,803 and EP286252A2, incorporated herein by reference.

Although all of the above disclosures show some cyanine dyes have anti-cancer activity, none of the prior art shows a correlation of anti-cancer activity with electrochemical reduction potential or shows the use of a series of dyes systematically varying in electrochemical reduction potential to identify useful anti-cancer agents.

Other publications have shown that some cyanine dyes show anti-cancer activity. See I. Minami, Y. Kozai, H. Nomura and T. Tashiro, Chem. Phar. Bull. 30:3106 (1982); W. M. Anderson, D. L. Delinick, L. Benninger, J. M. Wood, S. T. Smiley and L. B. Chen, Biochemical Pharmacology, 45:691 (1993). Dr. Lan Bo Chen at the Dana-Farber Cancer Institute examined the dyes listed in Table I and found that several of them showed encouraging in vitro anti-cancer activity. (See L. G. S. Brooker, L. A. Sweet, Science, 105, (1947) and I. Minami, Y. Kozai, H. Nomura, T. Tashiro, Chem. Pharm. Bull., 30,3106 (1982) on chemotherapeutic investigations of cyanine dyes.) Table I lists initial in vitro screening results in various cell lines and a comparison with Adriamycin, which is a widely used chemotherapeutic agent. The cyanine dyes were effective at much lower concentrations than Adriamycin and they showed excellent selectivity in their toxicity to cancer cells compared to normal cells.

TABLE I
The Initial Screening Results
IC₅₀ values (concentration at which 50% of the cells of the culture are killed.)

| | Cyanines | | | | Thiopyryliums | | | | Adriomycin |
|---|---|---|---|---|---|---|---|---|---|
| Compound | D-101 | D-160 | D-150 | O-1 | #10 | 7-1 | 6-27 | 6-49 | Adria |
| CV-1 Normal | 0.5 | 0.3 | 3 | 7.5 | 0.5 | 0.9 | 5 | 0.15 | 0.8 |
| Monkey Kidney | | | | | | | | | |
| Epithelial | | | | | | | | | |
| CX-1 | 0.005 | <0.005 | 0.03 | 0.5 | 0.15 | 0.18 | 0.015 | 0.015 | 0.55 |
| Human Colon | | | | | | | | | |
| Carcinoma | | | | | | | | | |
| MCF-7 | 0.03 | <0.005 | 0.03 | 0.3 | 0.03 | 0.05 | 0.05 | 0.05 | 0.05 |
| Human Breast | | | | | | | | | |
| Carcinoma | | | | | | | | | |
| CRL 1420 | 0.005 | <0.005 | 0.03 | 0.75 | 0.07 | 0.07 | 0.06 | 0.03 | 0.06 |
| Human Pancreatic | | | | | | | | | |
| Carcinoma | | | | | | | | | |
| EJ | <0.005 | <0.005 | 0.01 | 0.3 | 0.03 | 0.04 | 0.03 | 0.03 | 0.1 |
| Human Bladder | | | | | | | | | |
| Carcinoma | | | | | | | | | |
| CCL 185 | 0.01 | <0.005 | 0.02 | 0.8 | 0.05 | 0.05 | 0.1 | 0.05 | 0.07 |
| Human Lung | | | | | | | | | |
| Carcinoma | | | | | | | | | |
| SCC 68 | 0.03 | <0.005 | 0.1 | 0.3 | 0.04 | 0.06 | 0.3 | 0.03 | 0.08 |
| Human Squamous | | | | | | | | | |
| Cell Carcinoma | | | | | | | | | |
| CCL 105 | <0.005 | <0.005 | 0.01 | 0.3 | 0.03 | 0.03 | 0.03 | 0.007 | 0.05 |
| Human Adrenal | | | | | | | | | |
| Cortex Carcinoma | | | | | | | | | |

None of the prior art appears to have recognized that there is a strong correlation between the cyanine dyes that have the most powerful selective anti-cancer activity and their electrochemical reduction potentials. The publication by I. Minami, Y. Kozai, H. Nomura and T. Tashiro in Chem. Phar. Bull. 30:3106 states that “we could not find any definite correlation between the anti-cancer activity of cyanine dyes and their reduction potentials”. The authors do not identify the cyanine dyes that did not correlate or show any data to support this statement.

In the present study of over 2000 cyanine dyes, it was found that many dyes having electrochemical reduction potentials between −0.8 and −1.1V showed kill rates of only 10 or less. However, all of the cyanine dyes with selective kill ratios, that is, kill ratios which are higher for cancer cells than for healthy cells, of 60 or greater had electrochemical reduction potentials between—0.8 and −1.1V, without exception. This observation is believed to establish a definite correlation between anti-cancer activity of the cyanine dyes and their electrochemical potentials.

Previous studies may have been limited in the number of cyanine dyes studied and the availability of measured electrochemical reduction potentials. One would only have to evaluate the series of dyes offered for free to any investigator that was published in the Kodak advertisement (FIG. 1) to observe and confirm that the anti-cancer activity of cyanine dyes is strongly correlated with their electrochemical reduction potential.

### PROBLEM TO BE SOLVED

There remains a need for a pharmacological compound or composition having significant selective toxicity to at least some types of cancerous tissues, while not damaging normal tissues. In addition, there is a need for a method to identify compounds, for example cyanine dyes and merocyanine dyes, that have anti-cancer activity, so that the kill ratios are in excess of 60:1, may in excess of 100:1.

### SUMMARY OF THE INVENTION

The present invention relates to a method for selecting pharmacological compounds for selective inhibition of cancer cells comprising identifying a compound, determining the reduction potential (ER) of the compound, and selecting the compound which has a reduction potential from −1.1 to −0.8 volts. The invention also relates to a pharmacological compound comprising at least one cyanine dye or merocyanine dye, wherein the dye has at least one cationic substituent, wherein the dye has a reduction potential of from —1.1 to 0.8 volts, and wherein the pharmacological compound demonstrates selective inhibition of cancer cells.

### ADVANTAGEOUS EFFECT OF THE INVENTION

The present invention includes several advantages, not all of which are incorporated in a single embodiment. The cyanine and merocyanine dyes of the present invention demonstrate strong anti-cancer activity, with certain of the dyes showed very high anti-cancer activity with kill ratios in excess of 60:1. This invention will make it possible to identify compounds that have high anti-cancer activity based on their electrochemical reduction potential, which reduces the amount of testing that has to be done in biological systems, thereby reducing the cost of research and making it faster to identify anti-cancer agents. In one embodiment of the present invention, cyanine and merocyanine dyes demonstrate reduced toxicity to non-cancerous living cells, which could result in a lower incidence of deleterious side effects. Cyanine and merocyanine dyes are also more easily preparable in large scale, and a pyrogen-free, high purity preparation is obtainable at a low cost.

The active cyanine dyes screened were effective at much lower concentrations than the thiapyrylium dyes while retaining high selectivity, defined as the ability to kill cancerous cells without or with minimum effects on non-cancerous cells. Some of the dyes have good water solubility. There is also some evidence that the cyanines may be operating by a different mechanism than the rhodamine or thiapyryliums since they are effective against some cancer cell lines that the other dye classes are ineffective against.

### BRIEF DESCRIPTION OF THE DRAWINGS

FIG. 1 represents a list of 18 cyanine dyes varying in redox properties made available by Kodak to the scientific community in 1976.

FIG. 2 represents the correlation of dye reduction potential (Er) with the ability to kill human colon cancer cells relative to healthy cells.

FIG. 3 illustrates the IC50 in the CV cell line versus electrochemical reduction potential (Er).

FIG. 4. IC₅₀ in the CX cell line versus electrochemical reduction potential (Er), plotted CV/CX vs. Er.

FIG. 5. CV/CX versus electrochemical reduction potential (Er). * Points are Dyes with Chain Substituents.

FIG. 6 represents a plot of CV/CX versus π parameter for the thiacarbocyanine chromophore.

FIG. 7. Plot of CV/CX versus the MR parameter for the thiacarbocyanine chromophore.

FIG. 8. Plot of CV/CX versus the π parameter for the oxathiacarbocyanine chromophore.

FIG. 9. Plot of CV/CX versus the MR parameter for the oxathiacarbocyanine chromophore.

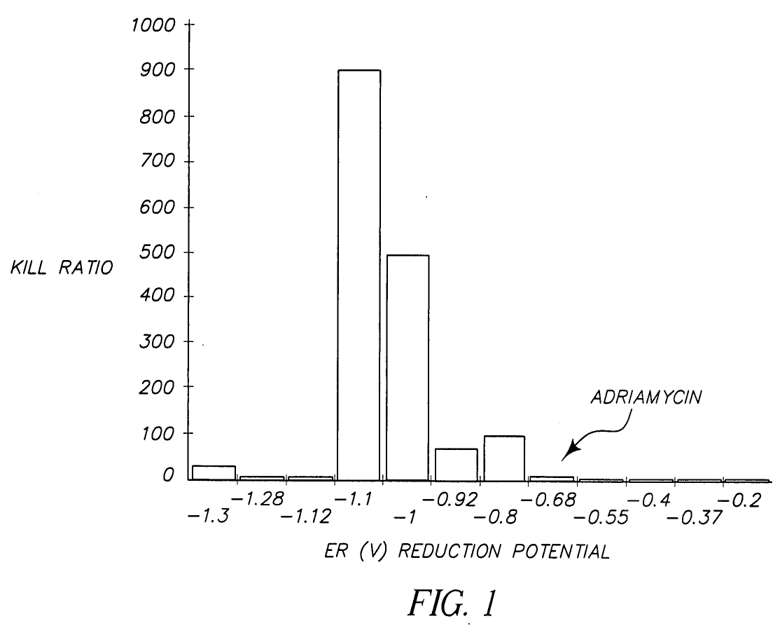

### DETAILED DESCRIPTION OF THE INVENTION

The present invention relates to a method of identifying compounds having selective toxicity to cancer cells. The process of the present invention, employing electrochemical reduction potential, may be used to identify compounds, which have very high anti-cancer activity, with kill ratios of 60:1 or greater. This process is especially useful for selecting photographic dyes to be used as anti-cancer agents, most preferably to identify cyanine and merocyanine dyes.

The electrochemical reduction potential of a dye has been shown to influence the ability of the dye to inhibit the growth of healthy sea urchin eggs in U.S. Pat. No. 4,226,868. Dyes which effectively inhibit the growth of sea urchin eggs were characterized by an electrochemical reduction potential equal to or more negative than −1.0 volt and by the ability to be absorbed by the cells of the eggs. These dyes were highly selectively toxic to differentiated carcinoma cells at unexpectedly low level of dilution. The ability of these dyes, particularly methine dyes, to inhibit the growth of sea urchin eggs did not appear to be related to the size, bulk, or molecular weight of the dye, except to the extent that such factors affected the ability of the of the dye to be absorbed by the cell, nor was it affected by substituents on the dye, except to the extent that each substituent affected the reduction potential of the dye. The oxidation potential of the dye did not correlate with its ability to inhibit growth.

However, certain trends in structure/activity have emerged, as a result of the work resulting in the present invention. In general, it appears that most active dyes for the inhibition of sea urchin eggs have electrochemical reduction potentials of from −0.80 to −1.1 V and are cationic. For cationic cyanine dyes, a number of factors affect dye selectivity. Chain length, substitution, symmetry, and aqueous solubility of the compound(s) are other factors to consider. Selectivity is defined as the ratio of CX/CV of IC₅₀ values in the CV and CX cell lines. Larger ratios are indicative of higher selectivity.

CX-1 is a cancerous human colon adenocarcinoma cell line. CV-1 is a non-cancerous control, which is a kidney epithelial cell line from an African green monkey. IC₅₀ is the dye concentration at which 50% of the cells are killed. Selectivity or kill ratio is defined as the ratio of CX1/CV-1 at IC₅₀ for both CX-1 and CV-1. A ratio with a high number indicates a high selectivity.

The first factor is the charge on the compound. A very large number of neutral and anionic compounds, particularly dyes, were screened and nearly all of them were inactive. Thus, the overwhelming observation is that a compound should be cationic or have a net positive charge in order to show significant biological activity. This is in accord with mechanistic proposals in which the cationic compound, particularly a dye, is drawn into the mitochondria because of the internal negative potential. Since the mitochondrial membrane potential in cancer cells is higher than that for normal cells, cationic dyes may be drawn into the cancer cells more readily. However, there were a few compounds that do not have a net positive charge but still show selectivity. These compounds may have anionic substituents which may be protonated during screening, resulting in a net positive charge.

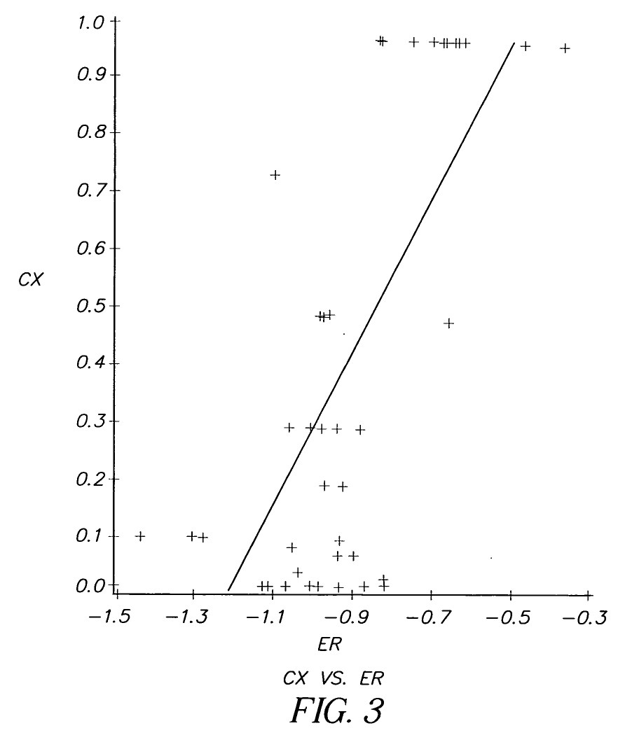

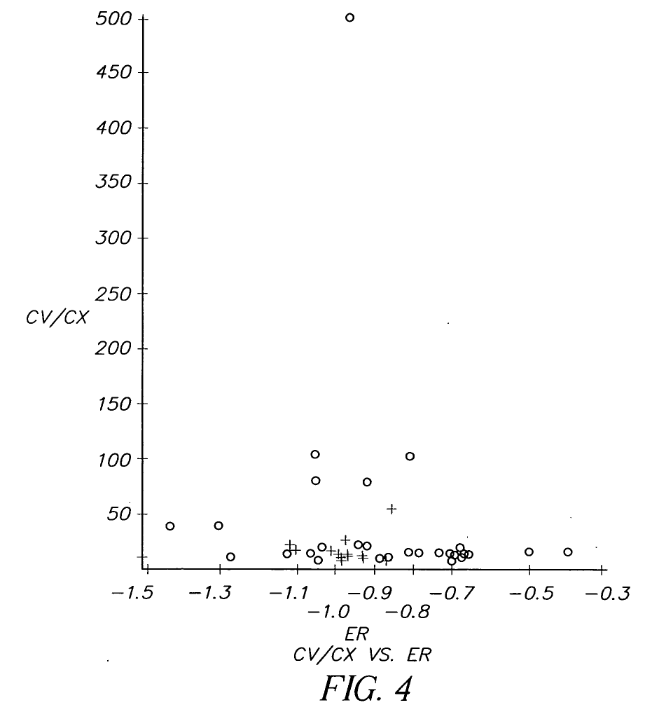

The next factor relates to electrochemical reduction potential (Er). There appears to be a linear correlation between the electrochemical reduction potential (Er) and toxicity. FIGS. 3 and 4 are plots of electrochemical reduction potential (Er) versus CX-1 (r=0.71) and CV-1 (r=0.72), respectively, where r is the correlation coefficient. As electrochemical reduction potential (Er) becomes more negative, the IC₅₀ values decrease (toxicity increases).

The mechanism of the observed anti-cancer activity of cyanine dyes with electrochemical reduction potentials between −0.80 and −1.1V is not known. It has been speculated that the anti-cancer activity of cyanine dyes may be related to an “inhibition of the respiratory chain reactions on the mitochondrial membrane of cells” (see I. Minami, Y. Kozji, H. Nomirro and T. Ijshiro, Chem. Phdfm. Bull. 30:3106(1982). One study suggested that the mode of biological action of cyanine dyes may be an “inhibitor of electron flow”. See K. W. Kinnally and H. Tedeschi, Biochemical and Biophysical Acts, 503:380(1978).

Another study concludes that the actions of one cyanine was involved with the inhibition of endogenous respiration of Eh1-lich acetes tumor cells. See E. Okimaso, J. Akiyama, N. Shiraishi and K. Ltsumi, Physiol. Chem. and Physics, 11:425 (1979).

Another study has discussed cessation of cell proliferation by adjustment of cell redox potential. (See A. Hoffman, L. M. Spetner and M. Burke, J. Theor. Biol. 211:403 (2001). No mention of cyanine dyes was made or the proposed use of a series of compounds varying in reduction potentials to test this theory.

It has also been disclosed that positively charged organic compounds may be selectively retained in cancer cells relative to healthy cells (See J. R. Wong and L. B. Chen, Radiation Oneology: Technology and Molecular Biology, Ed. P. Mauch, J. Loeffler, Published by W. B. Saunders P-300-315: S. D. Bernal, D. D. Burkett, R. E. Green, S. D. Rose, U.S. Pat. 2003/0017158A1 (2003): J. R. Wong, L. B. Chen, Advances In Cell Biology, 2: 263 (1988).

The present invention also showed that not only was the electrochemical reduction potential an important factor in anti-cancer activity but nearly all of the most effective dyes were also cationic, positively charged molecules.

The present results are consistent with the possibility that the cyanine dyes, with the highest kill ratio of cancer cells to healthy cells, absorb to some election donor sites and inhibit an electron transfer act important for cell growth.

The unexpected result was that the inhibition of an election transfer act was observed to be different for healthy cells than for cancer cells which allows for the observed very high selectivity which for some class of dyes was as high as 900 to 1, which is much greater than any known anti-cancer agent to date.

A possible site of action could be the mitochondrial membrane, where active electron transport occurs to support cellular respiration. A compound, such as a dye, with a electrochemical reduction potential (Er) more negative than the respiratory chain sites could occupy electron donor sites, creating an electronic barrier and inhibiting the transport of electrons and thus cellular respiration. If normal and cancer cells have different electron donor/acceptor properties, it might be that certain dyes with a reduction potential in the right range could create an electron barrier in cancer cells but not in normal cells.

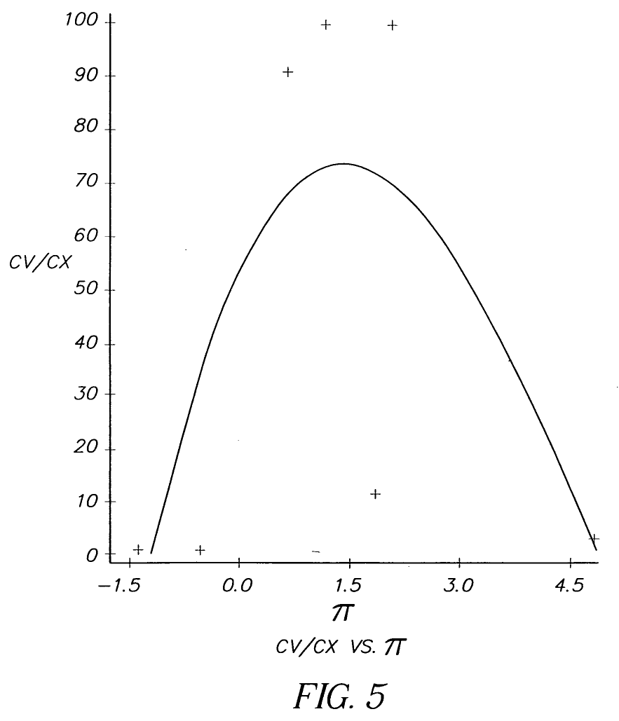

Dyes with an electrochemical reduction potential (Er) more negative than −1.00 V appear to be effective inhibitors, as disclosed in U.S. Pat. No. 4,226,868, whereas those with less negative electrochemical reduction potential (Er) values were not. The current data indicate that dyes with electrochemical reduction potential (Er) between −0.80 and −1.1 V may have the highest selectivity. Dyes with electrochemical reduction potential (Er) more negative than −1.1 V may inhibit electron transport in both normal and cancer cells and selectivity would be lost. Dyes with an electrochemical reduction potential (Er) more positive than −0.80 V may be relatively nontoxic to both cell lines. Electrochemical reduction potential (Er) is plotted versus CX-1/CV-1 in FIG. 5.

In the practice of this invention the reduction potential of a given dye may be determined in various ways. If available, the published reduction potential may be employed. Reduction potentials for numerous cyanine and merocyanine dyes are available in the literature. (See J. Lenhard, Journal of Imaging Science, vol. 30 pp. 27-35 (1986).) A large amount of data has been generated on the reduction and oxidation potentials of these dyes from their use in photography and related arts where they are employed, inter alia, as spectral sensitizers.

If a published reduction potential is not available, suitable techniques for measuring reduction potential are available. Accurate electrochemical oxidation and reduction potentials for these organic dyes can be measured by the techniques of fast scan cyclic voltommetry (CV) or second harmonic ac voltommetry (SHACV). SHACV measurements are made as described by Lenhard in the Journal of Imaging Science volume 30, 1986 pp 27-35 using a commercially available (Princeton Applied Research Corp.) potentiostat in conjunction with voltage programmer, a conventional lock-in amplifier, and a low-distortion oscillator. The SHACV method utilizes an ac voltage waveform (of a frequency greater than or equal to 400 hz) that is superimposed on a DC potential ramp. On the other hand, the measurement of the oxidation or reduction potential by the CV method utilizes a (high frequency) triangular voltage waveform. The general procedures for measuring potentials by this method have been described by Wightman in Analytical Chemistry, 1984, vol. 56, pp524 and Journal of Physical Chemistry, 1984, vol. 88, pp3915. The specific application of this technique to the measurement of cyanine dye potentials has been reported by Nomura and Okazaki, Chemistry Letters, 1990, pp2231-2234. Commercial instrumentation especially designed for fast scan (i.e., high frequency) CV is available. It is noteworthy that because of the complicated nature of many of the cyanine dyes, electrochemical reactions, conventional slow scan cyclic voltommetry or polarography methods (as described by R. Large in Photographic Sensitivity, R. Cox, ed., Academic Press, New York, 1973, pp 241-263) provide only approximate values for oxidation and reduction potentials. Errors as large as 0.4 V from the true potential can be obtained by using slow scan CV or electrochemical methods.

The procedure for measuring the oxidation or reduction potential by the SHACV or fast scan CV method utilizes solutions of acetonitrile (or similar nonaqueous solvent) with added tetrabutylammonium tetrafluoroborate TBABF₄ at a concentration of 0.1 M and ca. 5×10⁻⁴ M of the cyanine, oxonol, or merocyanine dye. For measurement of the reduction potential the solution must be deaerated with argon prior to examination. The preferred working electrode is a Pt disk (ca. 0.02 cm²) that is polished with 1 μm diamond paste, rinsed with water, and dried before each experiment. The reference electrode is a NaCl saturated calomel electrode. The cell and reference electrodes are maintained at 22 C. A value of 40 mV is added to the measured potentials to convert the number to that corresponding to an Ag/AgCl reference electrode. If a solvent other than acetonitrile is used then the measured potential must be referenced to a standard organic redox system such as ferrocene and corrected accordingly.

There are still a number of dyes that have the correct reduction potential but have low selectivity. This should not be surprising since many other factors may also influence selectivity.

Another factor to consider is the chain length of the compound. For example, the length of the methine chain in a cyanine dye has a large influence on the wavelength of absorption of the dye and its redox properties. It also, of course, determines the separation distance between the two heterocycles. Since chain substitution may lower selectivity, this may affect the correlation.

Yet another factor to consider is the substituents on the compound. Substitution on the nitrogen atoms of a heterocycle may affect steric interactions and may also influence the net hydrophobicity of the compound. In one preferred embodiment, methyl or ethyl substitution on nitrogens, especially when the compound contains two nitrogens, generally results in the highest selectivity. However, it is possible to place certain substituents other than methyl or ethyl on one of the nitrogens and retain high selectivity. Chain substitution may also work to decrease selectivity, although not necessarily activity. The chain substituent would be expected to affect steric, electronic and hydrophobicity factors. It could also change the conformation of the chain from transoid to cisoid. Dyes with meso thio-alkyl substituents in some cases retain good, although lower selectivity. Also, for certain dyes, back-ring substitution may increase selectivity. If intercalation is important, then hydrogen-bonding substituents should increase selectivity, as disclosed in H. W. Zimmerman, Angew. Chem. Int. Ed., 25, 115-130 (1986) incorporated herein by reference. A more detailed analysis of benzothiazole and benzoxazole-containing carbocyanines using a quantitative structure activity approach suggests that dye selectivity is dependent on both the hydrophobicity and steric parameters of the nitrogen substituents. Dyes which have small nitrogen substituents having a hydrophobicity parameter within a certain range give the highest selectivities.

It appears that, for the case of cyanine dyes, carbocyanines (3 methine carbon chain) which do not have substitution on the methine chain in general give the highest selectivity values. The nature of the heterocyclic nuclei in the dye as well as the type of substituent on the nitrogen atoms is also critical.

Dye symmetry also appears to be a factor to consider. Some symmetrical compounds, such as dyes containing two indole nuclei and dyes containing two benzothiazole nuclei have high selectivity. However, for some types of compounds, such as dyes containing the benzothiazole nucleus and various heterocycles as the other nucleus when the second nucleus is benzoxazole or isoindole, lack of symmetry produces higher selectivity than the symmetrical compounds, for example thiacarbocyanine.

Dye solubility is also a factor, since compounds having low water solubility are difficult or impossible to include in in vivo testing.

The preferred compounds for identification utilizing the present invention are methine dyes, which comprise a methine chain, that is, a chain of carbon atoms with alternating double and single bonds) terminated at each end with a hetero atom. The terminal heteroatoms are typically nitrogen or oxygen atoms in various combinations, and generally they are contained in or attached to an unsaturated cyclic nucleus. Typical methine dyes are cyanine dyes, merocyanine dyes and oxonol dyes.

The most preferred class of compounds for identification utilizing the present invention may include cyanine dyes and merocyanine dyes. A cyanine dye has two basic nuclei connected by a conjugated chain having an odd number of methine carbons. A merocyanine dye has one basic nucleus and one acidic nucleus separated by a conjugated chain having an even number of methine carbons. Basic and acidic nuclei are discussed in The Theory of the Photographic Process, 4^{th} edition, T. H. James, editor, Macmillan Publishing Co., New York, 1977. In one embodiment the cyanine dye of the invention is represented by Formula 1a.

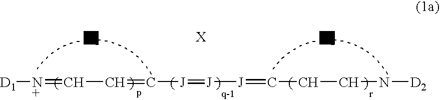

In Formula 1a, E₁ and E₂ may be the same or different and represent the atoms necessary to form a substituted or unsubstituted heterocyclic ring which is a basic nucleus. For example, E₁ and E) may independently represent a substituted or unsubstituted benzothiazole group, benzoxazole group, indole group, or benzimidazole group.

Each J independently represents a substituted or unsubstituted methine group. For example, substituents may be an aryl group, such as a phenyl group or an alkyl group, such as a methyl or ethyl group. In one suitable embodiment, J represents an unsubstituted methane group.

Each q is a positive integer of from 1 to 4. In one desirable embodiment, q is 2. In Formula 1a, p and r each independently represents 0 or 1. In one desirable embodiment, p and r each represent 0.

D₁ and D₂ each independently represent a substituted or unsubstituted alkyl or substituted or unsubstituted aryl group. In one suitable embodiment D₁ and D₂ each independently represent an independently selected alkyl group.

In Formula 1a, X represents one or more pharmaceutically acceptable anions. The term “pharmaceutically acceptable anion” for X, which balances the electrical charge in the compounds above, is intended to mean an ion, when administered to the host subjected to the method of treatment of this invention, which is non-toxic and which renders the compounds above more soluble in aqueous systems.

In one embodiment, the merocyanine dye of the invention is represented by Formula 1b.

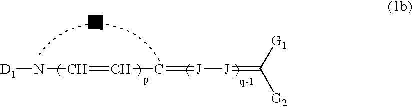

In Formula 1b, E₁, D₁, J, p, q are as defined above for formula (1a). G₁ and G₂ represent an electron-accepting group such as a cyano group or carboxyethyl group. In Formula 1b, G. and G₂ may combine to form a ring that comprises an acidic nucleus. For example, they may combine to represent the atoms necessary to complete a barbituric acid, rhodanine, hydantoin, idanedione, isoxazolone, or pyrazolidinedione nucleus.

In another suitable embodiment, the merocyanine dye of the invention is represented by Formula 1c.

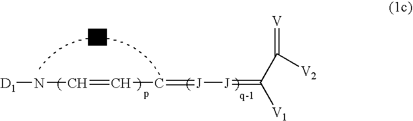

In Formula 1c, E₁, D₁, J, p, q are as defined above for formula (1a), V represents an electron-accepting atom or group, such as O, S, or C(CN)₂, V₁ represents an electron-withdrawing, for example a cyano group, V₂ also represents an independently selected substituent, such as a phenyl group. In Formula 1c, V₁ and V₂ may combine to form a ring that comprises an acidic nucleus, for example, they may combine to represent the atoms necessary to complete a barbituric acid, rhodanine, hydantoin, idanedione, isoxazolone, or a pyrazolidinedione nucleus.

Preferred cyanine or merocyanine dyes have the nitrogen heteroatom, which terminates the methine chain in a heterocyclic nucleus. Typical nuclei are quinoline, pyridine, isoquinoline, 3H-indole, benzindole, oxazole, thiazole, selenazole, imidazole, benzoxazole, benzothiazole, benzoselenazole, benzimidazole, naphthothiazole, naphthoxazole, naphthoselenazole, pyrylium, and imidazolepyrizine. These nuclei are typically in the form of quaternary salts and are joined to one another by a methine chain containing an odd number of carbon atoms so that the nitrogen atoms are conjugated to one another (i.e., separated by alternating double and single bonds).

Suitable examples of pharmaceutically acceptable anions represented by X include halides such as chloride, bromide and iodine, sulfonates such as aliphatic and aromatic sulfonates, e.g., methanesulfonate, trifluoromethanesulfonate, p-toluenesulfonate, naphthalenesulfonate, 2-hydroxyethanesulfonate, and the like, sulfamates such as cyclohexanesulfamate, sulfates such as methyl sulfate and ethyl sulfate, bisulfates, borates, alkyl and dialkyl phosphates such as diethyl phosphate and methylhydrogen phosphate, pyrophsophates such as trimethylpyrophosphate and diethyl hydrogen pyrophosphate, carboxylates, advantageously carboxy- and hydroxy-substituted carboxylates and carbonates. Preferred examples of pharmaceutically acceptable anions include chloride, acetate, propionate, valerate, citrate, maleate, fumarate, lactate, succinate, tartrate and benzoate.

In one embodiment the methine dye is substituted with at least one cationic substituent. For example, the substituent may be a tetrasubstituted ammonium group, such as that represented by Formula (C-2). In Formula (C-2), each R₁, R₂, and R₃ independently represents a substituted or unsubstituted alkyl group, or a substituted or unsubstituted aryl group and provided that two of R₁, R₂, and R₃ may join together to form a ring. For example, each of R₁, R₂, and R₃ may independently represent groups such as a methyl group, an ethyl group, a 2-hydroxyethyl group, or phenethyl group. L represents a linking group that connects the cationic group to the dye chromophore. L may represent a substituted or unsubstituted methylene chain of at least two carbon atoms or L may represent a divalent aromatic group. For example, L may represent groups such as a trimethylene group or a tetramethylene group, or a phenylene group. L may contain heteroatoms, for example L may represent groups such as —CH₂CH₂OCH₂CH₂— and —CH₂CH₂OCH₂CH₂OCH₂CH₂—.

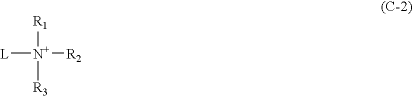

In another embodiment the methine dye is substituted with at least one cationic group represented by Formula (C-3). In Formula (C-3), L represents a linking group as described previously. Ar represents the atoms necessary to complete an aromatic group, which may be further substituted. For example, Ar may represent the atoms necessary to complete a substituted or unsubstituted pyridinium ring, quinolinium ring, or benzothiazolium ring.

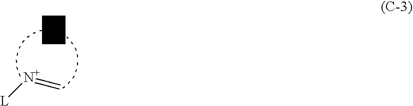

Methine dyes with cationic substituents are known for use in photographic applications, as disclosed in U.S. Pat. Nos. 6,620,581, 6,558,893, 6,361,932, 6,331,385, 6,329,133, 6,312,883, 6,165,703, and 6,143,486, all incorporated herein by reference.

Representative cyanine dyes include:

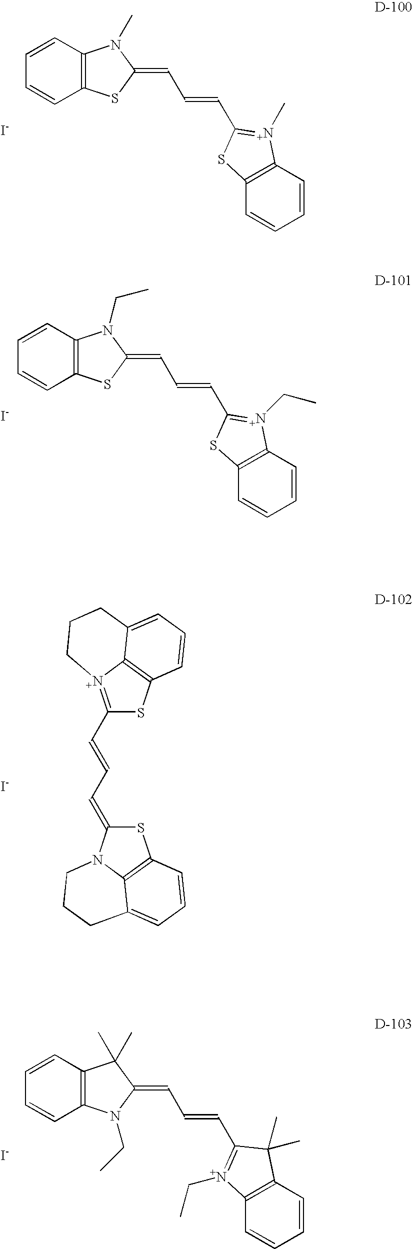

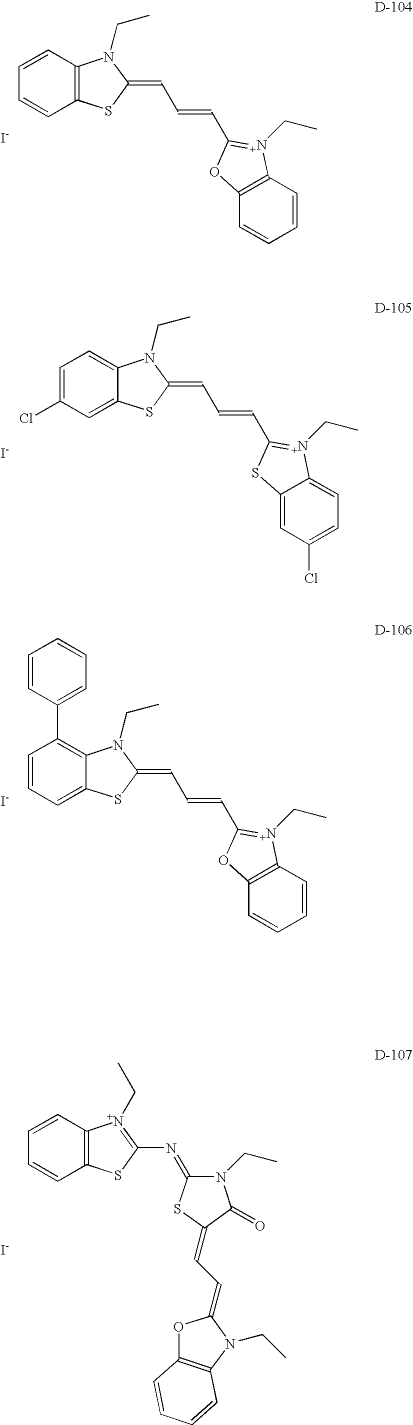

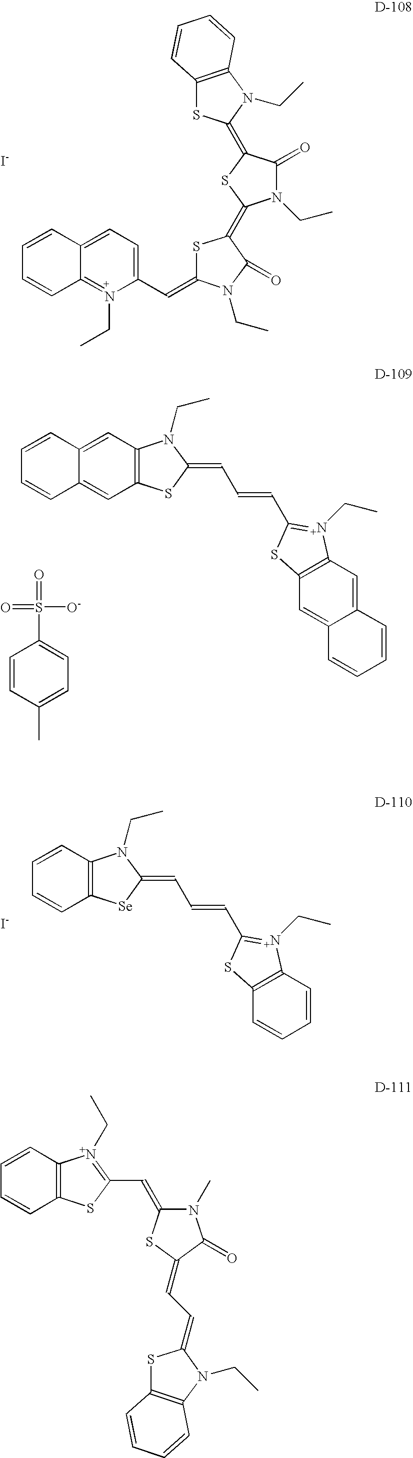

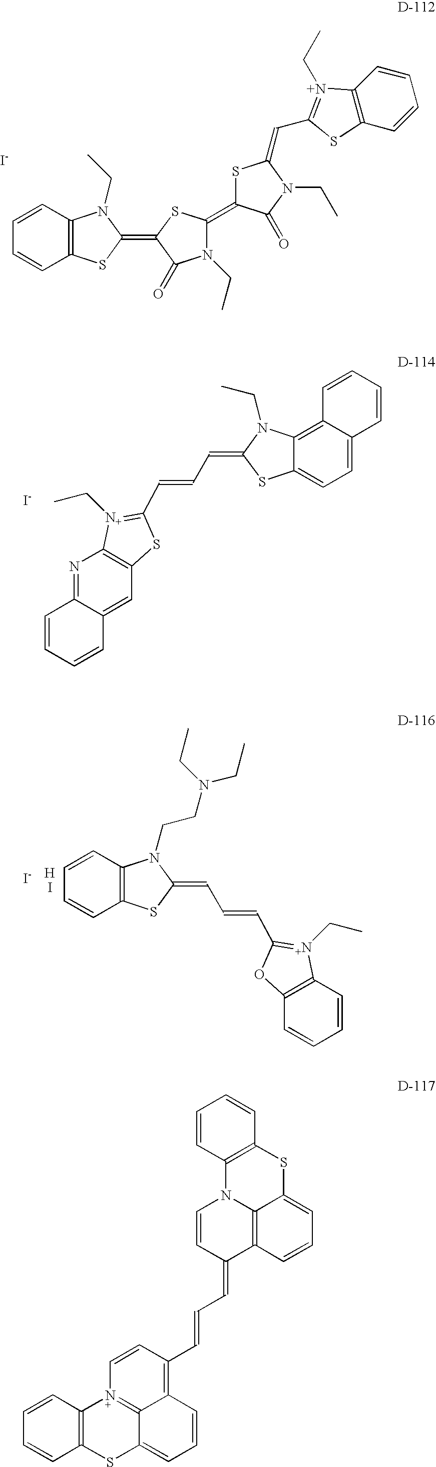

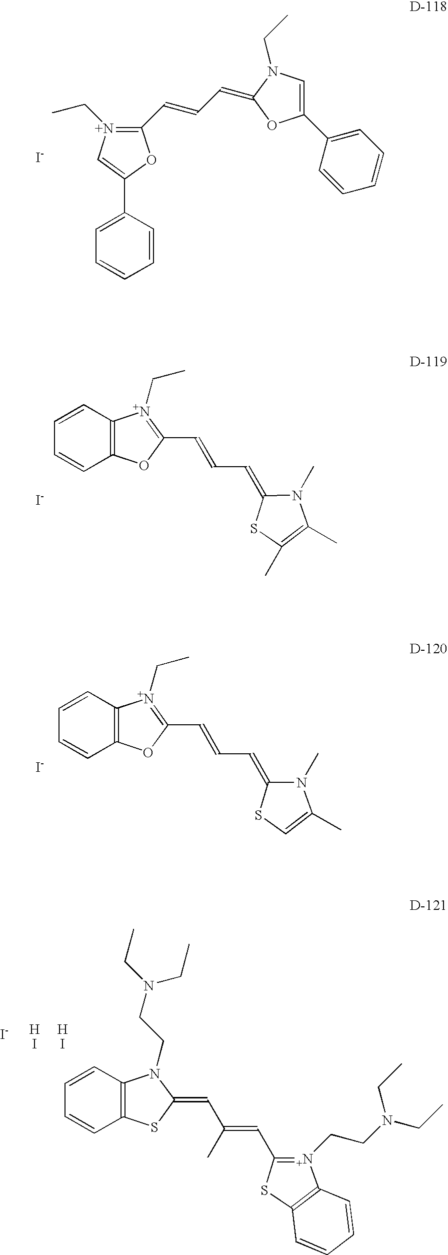

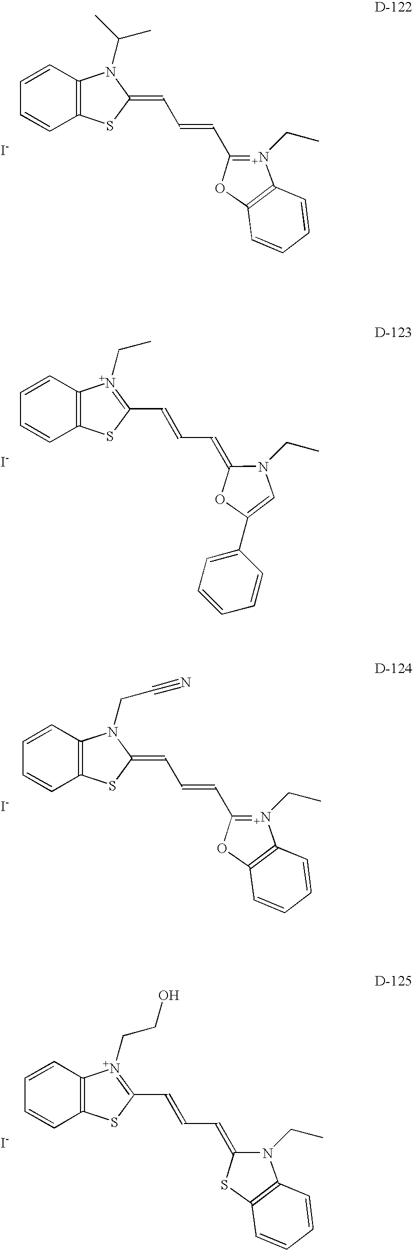

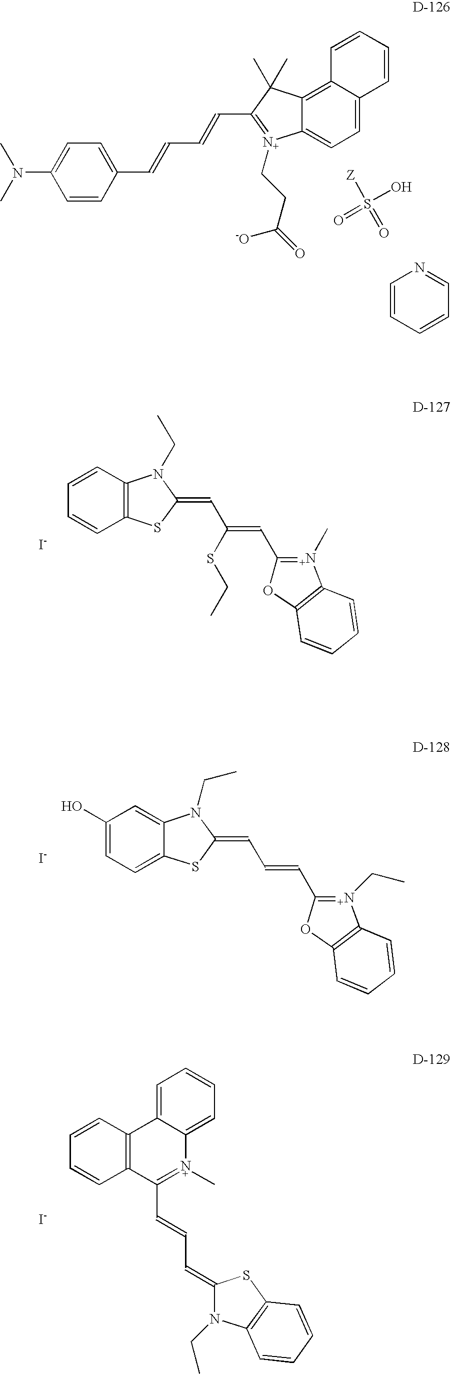

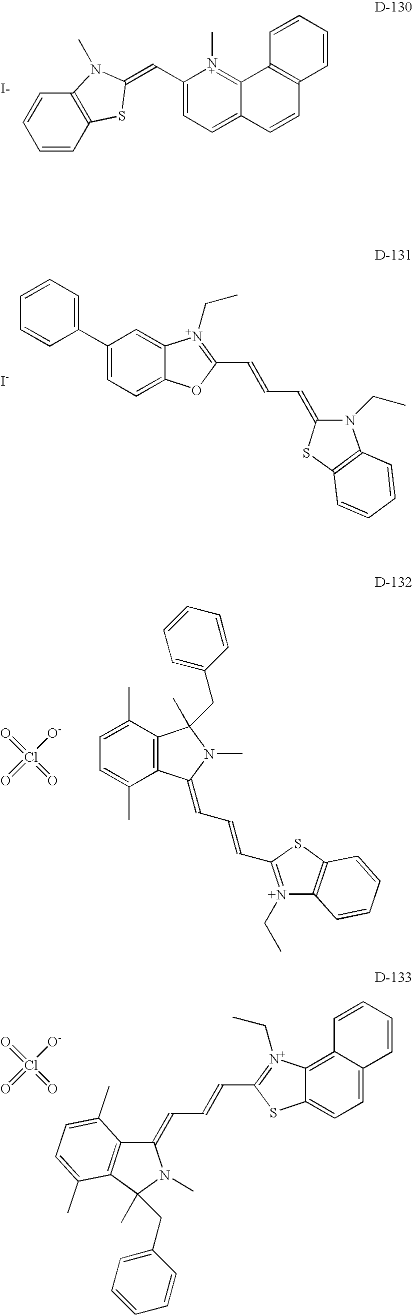

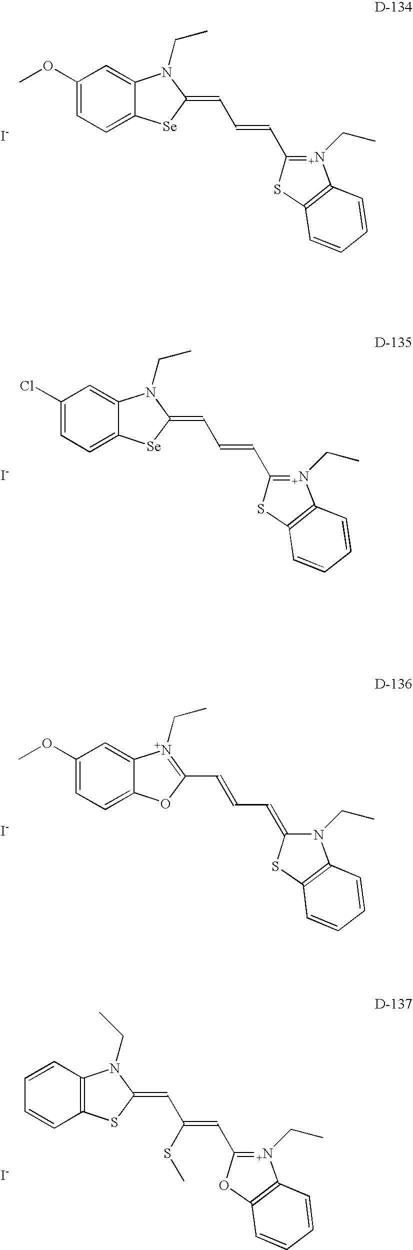

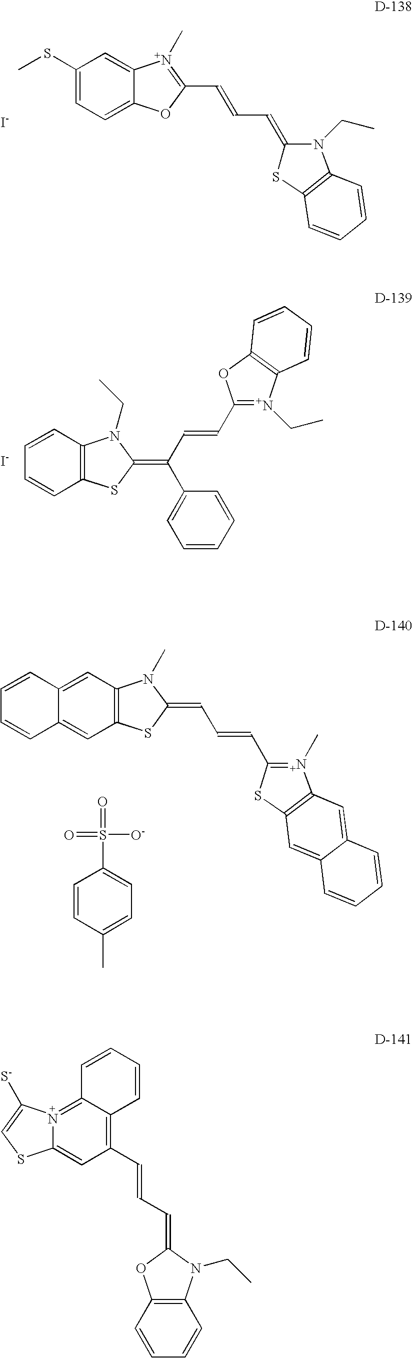

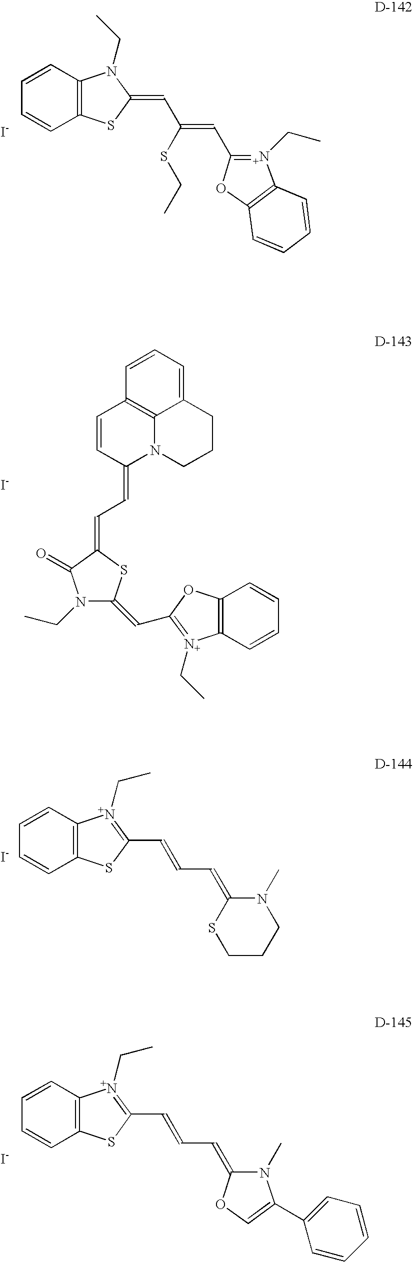

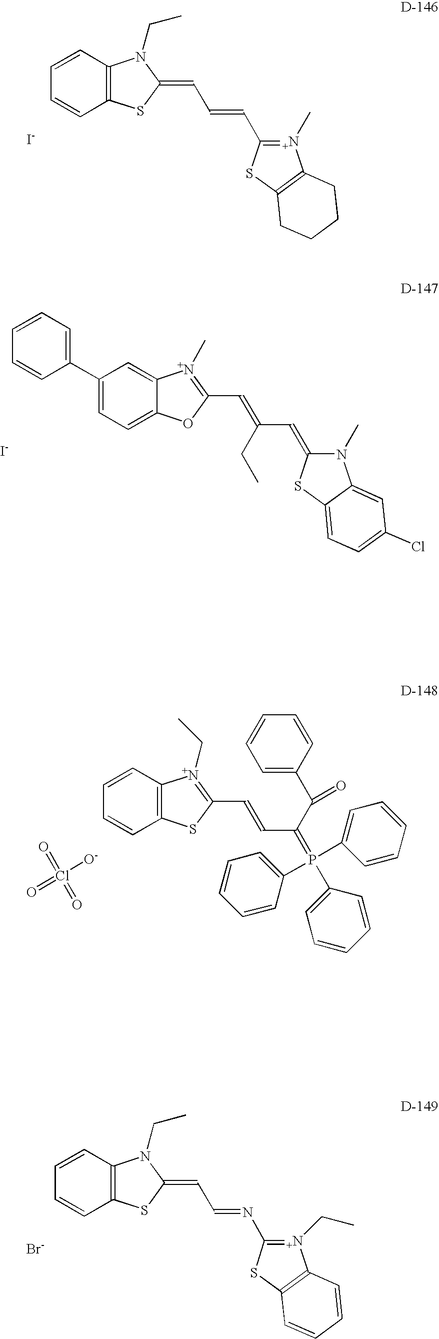

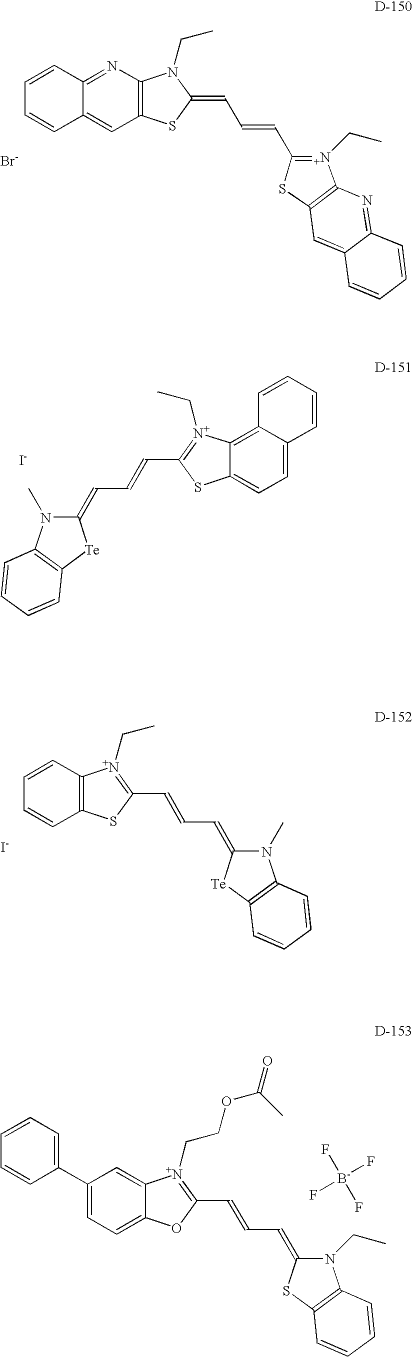

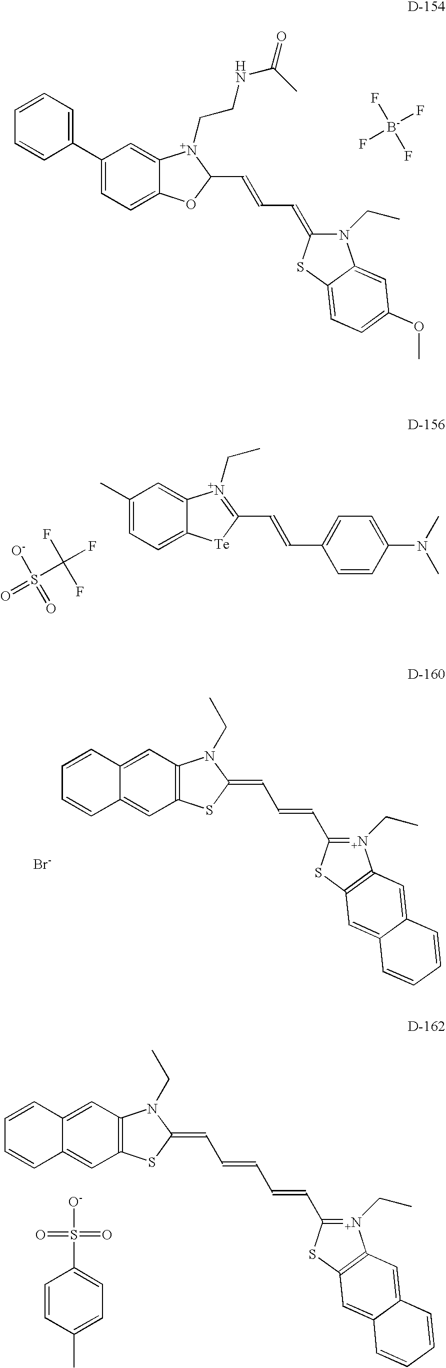

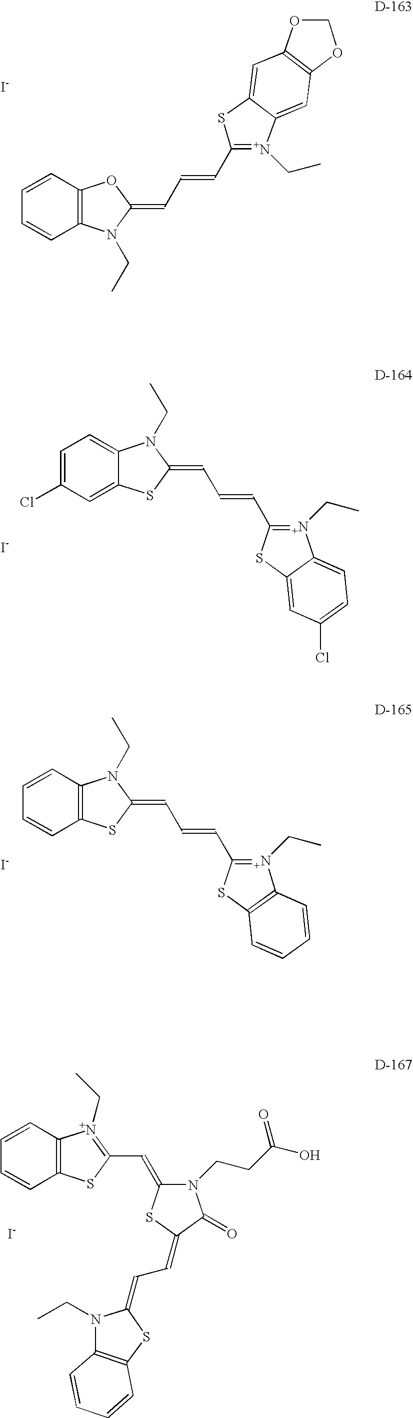

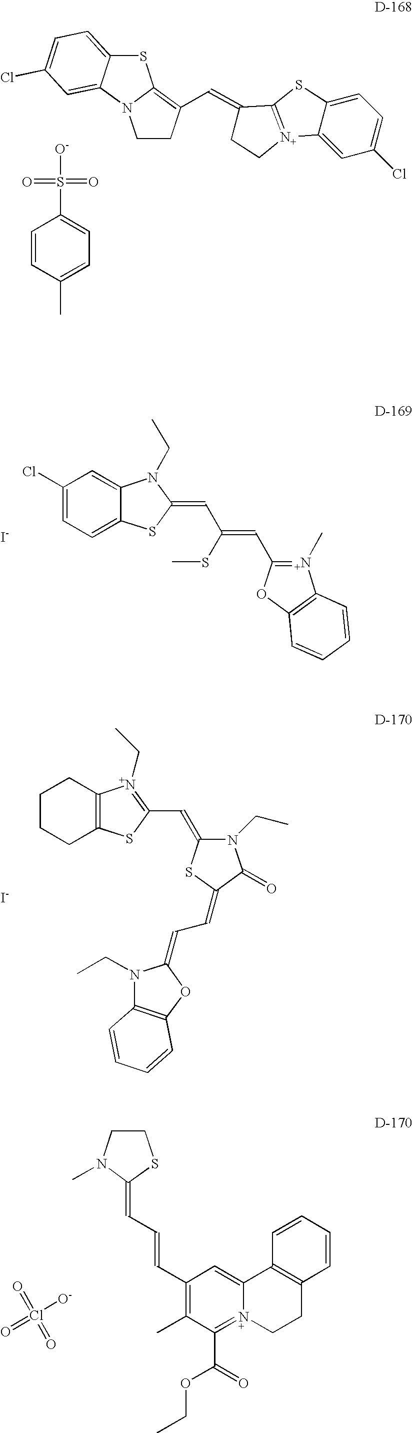

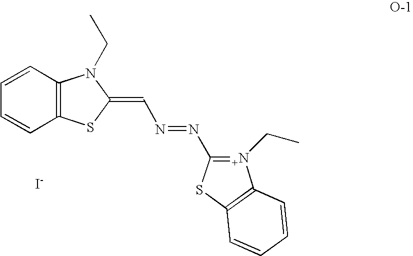

S13 is one of the simplest cyanine dye structures possible (in this nomenclature the S refers to the sulfur, the 1 indicates one carbon on the nitrogen and 3 indicates a 3 methine chain). One of its advantages is good solubility in 5% dextrose/water solutions, which would make it easy to administer. A synthetic challenge will be to enhance the activity/selectivity of this dye and retain its good water solubility.

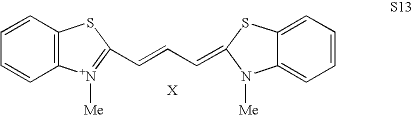

As discussed above, solubility of anti-cancer agents is a factor in their effectiveness for use in pharmacological situations. There are various methods known which may be used to increase solubility. For example, one method proposes a method for introducing a hydroxyl group and methoxyethyl group into the molecular structure of each compound, as disclosed in Japanese Patent Unexamined Published Application Nos. Sho 63-228, 145, 63-123,054 and 63-280,243, and Hei 3-168,634,4-145,431 and 1-196, 032, and European Patent No. 318,936. However, this often affords only limited aqueous solubility. The introduction of a sulfoalkyl group is another commonly used technique to increase solubility. For example, a 2-sulfoethyl or 3-sulfopropyl group can be attached to the dye. U.S. Pat. Nos. 5,599,825 and 5,476,945 disclose the addition of a nitrogen-containing heterocyclic group to increase solubility.

Although the addition of sulfoalkyl groups to a cyanine dye can greatly enhance its water solubility, the net charge of the dye is also changed. This is illustrated for dye S-13 in Table A. The addition of one sulfo group affords a zwitterionic dye (example A-2) having a net charge of zero. Zwitterionic materials are often very insoluble in both water and organic solvents. Addition of two sulfo groups yields an anionic dye (A-3). Anionic dyes have enhanced water solubility but will have difficulty entering the cell membrane due to their negative charge. Thus, it is a problem to be solved to increase the solubility of the dye while maintaining a net positive charge.

An alternative method of dye solubilization that solves this problem is to use one or more cationic substituents, for example, 3-(N,N,N-trimethylammonium)propyl or 3-(N,N,N-triethylammonium)propyl substituents. Dyes A-4, A-5, and A-6 in Table A, for example, have such substituents. These dyes will have enhanced aqueous solubility relative to dye A-1 but still have a net positive charge.

Likewise, in the case of merocyanine dyes, the chromophore has no net charge and low aqueous solubility, for example see Table A, dye A-7. One can increase the solubility by adding an anionic solubilizing groups (dye A-8) but the net charge of the dye is then −1. Adding a cationic substituent group, as in dye example A-9, affords both increased solubility and a positive charge.

In one embodiment, at least one of D₁ and D₂ of formula 1a includes a tetravalent nitrogen atom. In another embodiment D₁ of formula 1b includes a tetravalent nitrogen atom. Suitably the tetravalent nitrogen can be a trialkylammonium salt wherein the alkyl groups may be substituted or unsubstituted. The tetravalent nitrogen may be in an aromatic ring in which the positive charge is delocalized. Illustrative examples of D₁ and D₂ are shown below.

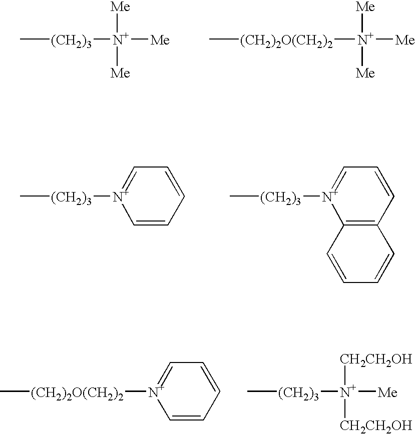

TABLE A
Effect of Substituent Groups on Dye Charge

| Example | Structure | Net Charge |
|---|---|---|
| A-1 | 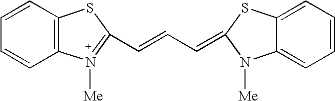 | positive 1 |
| A-2 | 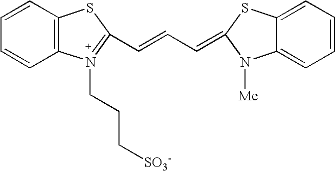 | zero |
| A-3 | 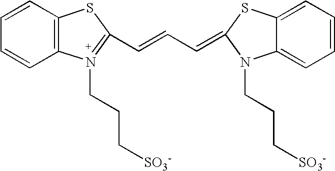 | negative 1 |
| A-4 | 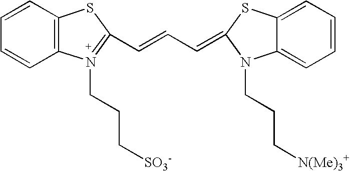 | positive 1 |
| A-5 | 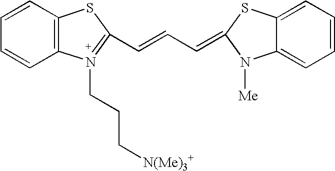 | positive 2 |
| A-6 | 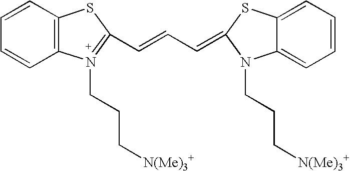 | positive 3 |
| A-7 | 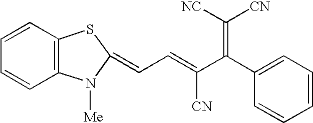 | zero |
| A-8 | 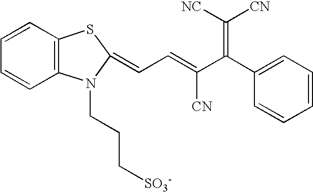 | negative 1 |
| A-9 | 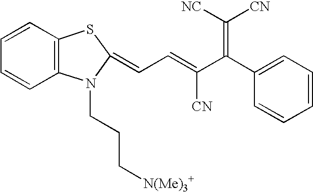 | positive 1 |

Of the compounds found to have enhanced activity, compounds D-140 and D-100 are most desirably soluble.

In one preferred embodiment, the pharmacological compound or compounds may be in the form of a composition effective to treat differentiated carcinoma or melanoma cells contained in a host mammalian body, comprising a therapeutically effective amount of the pharmacological compound in a carrier.

An acceptable carrier can be any carrier, such as a solvent that will sufficiently dissolve the pharmacologically active compound. Water alone may be a good carrier solvent, if the pharmacological compound is soluble in water. Water may also be selected if the pharmacological compound can be highly dispersed, for example, by sonication, into a fine suspension. Among preferred examples of a suitable carrier solvent for human usage are a 5% dextrose solution in water, or a mixture of ethanol and a polyol such as polyethoxylated caster oil, available from the National Cancer Institute as “Diluent No. 12”. Still other acceptable carrier solvents include, dimethyl sulfoxide (DMSO) for intravesical treatment, and isotonic saline for IV and IP injections.

Still other carriers that are useful may include materials such as gelatin, natural sugars such as sucrose or lactose, lecithin, pectin, starch, (for example cornstarch), alginic acid, tylose, talc, lycopodium, silica (for example colloidal silica), glucose cellulose, cellulose derivatives, for example cellulose ethers in which the cellulose hydroxyl groups are partially etherfied with lower aliphatic alcohols and/or lower saturated oxyalcohols (for example, methyl hydroxypropyl cellulose, methyl cellulose, hydroxyethyl cellulose), stearates, e.g. methyl stearate and glyceryl stearate, magnesium and calcium salts of fatty acids with 12 to 22 carbon atoms, especially saturated acids (for example, calcium stearate, calcium laurate, magnesium oleate, calcium palmitate, calcium behenate and magnesium stearate), emulsifiers, oils and fats, especially of plant origin (for example, peanut oil, castor oil, olive oil, sesame oil, cottonseed oil, corn oil, wheat germ oil, sunflower seed oil, cod-liver oil), mono-, di-, and triglycerides of saturated fatty acids (C₁₂ H₂₄ O₂ to C₁₈ H₃₆ O₂ and their mixtures), e.g., glyceryl monostearate, glyceryl distearate, glyceryl tristearate, glyceryl trilaurate), pharmaceutically compatible mono-, or polyvalent alcohols and polyglycols such as glycorine, mannitol, sorbitol, pentaerythritol, ethyl alcohol, diethylene glycol, triethylene glycol, ethylene glycol, propylene glycol, dipropylene glycol, polyethylene glycol 400, and other polyethylene glycols, as well as derivatives of such alcohols and polyglycols, esters of saturated and unsaturated fatty acids (2 to 22 carbon atoms, especially 10 to 18 carbon atoms), with monohydric aliphatic alcohols (1 to 20 carbon atoms alkanols), or polyhydric alcohols such as glycols, glycerine, diethylene glycol, pentaerythritol, sorbitol, mannitol, ethyl alcohol, butyl alcohol, octadecyl alcohol, etc., e.g. glyceryl stearate, glyceryl palmitate, glycol distearate, glycol dilaurate, glycol diacetate, monoacetin, triacetin, glyceryl oleate, ethylene glycol stearate, esters of polyvalent alcohols that are etherified, benzyl benzoate, dioxolane, glycerin formal, tetrahydrofurfuryl alcohol, polyglycol ethers of 1 to 12 carbon atom alcohols, dimethyl acetamine, lactamide, lactates, e.g., ethyl lactate, ethyl carbonate, silicones (especially medium viscosity dimethyl polysiloxane), magnesium carbonate and the like.

The composition may also contain other additives, and methods of preparation of the composition can be found in the extant literature, for example, U.S. Pat. No. 4,598,091 issued on Jul. 1, 1986.

The effective treatment of differentiated carcinomas includes regression, palliation, inhibition of growth and remission of tumors. The kind of specific organ cancers treatable by this invention include carcinomas of lung (except for those noted above), colon, breast, bladder, prostate, pancreas, stomach, vagina, esophagus, tongue, nasopharynx, liver, ovary, and testes.

The method of delivery of the dye includes implanted drug pump, intravenous (IV) intraperitoneal (IP) and intravesical injection, using a pharmaceutically acceptable carrier solvent.

The dosage levels depend upon which selective compound is being used on which differentiated carcinoma. Such dosage may be determined by one skilled in the art, using the techniques described in Goodman and Gilman's. “The Pharmacological Basis of Therapeutics” (6th edition), page 1675-1737, subtitled “Design and Optimization of Dosage Regimens” (Macmillan Publishing Co., New York, 1980).

Approximately 2000 dyes of a wide variety were screened for anti-cancer activity by using the following Dye Screening procedure.

### EXAMPLES

The following examples are provided to illustrate the invention.

Reduction Potential Measurement

SHACV (Second Harmonic Alternating Current Voltametry) measurements are made as described by Lenhard in the Journal of Imaging Science volume 30, 1986 pp 27-35 using a commercially available (Princeton Applied Research Corp.) potentiostat in conjunction with voltage programmer, a conventional lock-in amplifier, and a low-distortion oscillator. The SHACV method utilizes an ac voltage waveform (of a frequency greater than or equal to 400 hz) that is superimposed on a DC potential ramp.

The procedure for measuring the oxidation or reduction potential by the SHACV method utilizes solutions of acetonitrile (or similar nonaqueous solvent) with added tetrabutylammonium tetrafluoroborate TBABF₄ at a concentration of 0.1 M and ca. 5×10⁻⁴ M of the cyanine, oxonol, or merocyanine dye. For measurement of the reduction potential the solution must be deaerated with argon prior to examination. The preferred working electrode is a Pt disk (ca. 0.02 cm²) that is polished with 1 μm diamond paste, rinsed with water, and dried before each experiment. The reference electrode is a NaCl saturated calomel electrode. The cell and reference electrodes are maintained at 22 C. A value of 40 mV is added to the measured potentials to convert the number to that corresponding to an Ag/AgCl reference electrode. If a solvent other than acetonitrile is used then the measured potential must be referenced to a standard organic redox system such as ferrocene and corrected accordingly.

Dye Screening

A. Screening Procedure

The dyes were screened in two cell lines. CX-1 is a human colon adenocarcinoma cell line. CV-1 is a kidney epithelial cell line from an African green monkey. CV-1 was used as a reference because no normal human epithelial cell line is available for clonogenic assay.

In the initial screen, dyes were examined at six different concentrations to generate a dose response curve. An IC₅₀ value, the concentration at which 50% of the cells are killed, was then calculated. Initially, a dye concentration of 1.0 μg/mL was the highest examined for the CX-1 cell line and 0.005 μg/mL was the lower limit. The corresponding limits on the CV-1 cell line were 10.0 and 005 μg/mL.

The screening procedure used a response curve that was determined from eight dye concentrations. The upper and lower limits of CX-1 were 0.32 and 0.0015 μg/mL, respectively, and the corresponding revised limits on CV-1 were 3.2 and 0.025 μg/mL. The screening results were reported by assigning an IC₅₀ value to each dye for the CV-1 and CX-1 cell lines. These values will be referred to herein simply as CV and CX. The most desirable result would be to have a dye that was highly toxic to carcinoma cells but nontoxic to ‘normal’ cells. One measure of this type of selectivity is the ratio of the IC₅₀'s in the normal cell line to that in the carcinoma line, CV/CX.

Assay Procedure

Compounds are screened via an in vitro assay. CV-1 and CX-1 cells are seeded into separate assay plates. The plates are treated with varying concentrations of test compounds, rinsed, and colonies allowed to grow. IC₅₀'s are determined and drugs evaluated based on their selectivity and toxicity. Assay Steps:
I. Seeding Cells
II. Weighing
III. Preparation of Solution
IV. Treatment
V. Rinsing and Feeding
VI. Fixing
VII. IC₅₀ Determination
   I. Seeding Cells

The cells should be seeded 16-30 hours before treatment. The media for CV-1 cells is DME+5% calf serum +5% Nuserum. The media for CX-1 cells is RPMI/DME (50/50)+5% calf serum +5% Nuserum.

1. Determine:

(a) how many 48-well plates are needed based on 4 compounds per plate, 2 cell types per compound and duplicates for everything.

(b) volume of media needed based on 0.4 ml media/well.

(c) number of cells based on 2000 CX-1 cells/well and 1000 CV-1 cells/well. NOTE: Always make enough cell suspension for 1 extra plate to allow for volume loss during transfers.

2. Trypsinize cells for 3-5 minutes. Take them up in a few mls. Of media and pipet up and down to obtain a single cell suspension.

3. Count the cells.

4. Divide the number of cells needed, as determined in step 1 (c), by the number of cells you have. Take up this volume of cell suspension and dilute with media to the proper volume, determined in step 1 (b).

5. Using a repeating pipetter, aliquot 400 μl of cell suspension into each well. Agitate the suspension before each aliquot to insure an even suspension.

6. Incubate the plates at 37C.

II. Weighing

1. Autoclave vials, weighing paper and DMSO

2. Label the vials with our number (i.e. 3-54 for Batch 3, compound 54).

3. At the Mettler balance: Place the labeled vial (without top) on the Mettler and record weight. Shake 2-4 mg of the compound into it and weigh again. Subtract the original weight from the final and record this amount on the label of the vial.

4. The weighed compounds are kept at 4° C., wrapped with foil.

III. Preparing Solutions

1. Just prior to the start of the assay add sterile DMSO with a pipet to each vial of pre-weighed compound to make a 1 mg/ml solution.

2. Cap and shake thoroughly. If there is a precipitate, sonicate for −5-15 minutes, and record the solubility status.

3. Prepare the two concentrations needed per compound as follows:

(a) For CV-1: 256 ug/ml-256 ul of 1 mg/ml stock +744 ul DMSO
(b) For CX-1: 25.6 ug/ml-100 ul of 256 ug/ml+900 ul DMSO
   IV. Treatment
1. Each plate is treated with 4 test compounds, one positive control and one negative control.
2. Serial dilutions of the compounds are made in the following way:

2 multi-well pipetters are used. One is set for 10 ul and one for 200 ul. Each is equipped with 6 tips spaced such that they fit into the 6 rows of a plate. 390 ul of media is added to the first column of each plate. 10 ul each of 4 compound solutions, a positive and a negative control are taken up and put into the first column of wells. The 200 ul pipetter is then placed in the same wells, pipetted up and down 3 times and two 200 ul aliquots are moved to the next column of wells. This column is pipetted up and down 3 times and 400 ul moved to column 3, etc. The final 400 ul excess from column 8 is discarded.

| | Column | | | | | | | |
|---|---|---|---|---|---|---|---|---|
| | 1 | 2 | 3 | 4 | 5 | 6 | 7 | 8 |
| Volume | 400 | 400 | 400 | 400 | 400 | 400 | 400 | 400 |
| Cells/wellCX-1 | 2000 | 2000 | 2000 | 2000 | 2000 | 2000 | 2000 | 2000 |
| Final Conc.drug (ug/ml) | 0.32 | 0.16 | 0.08 | 0.04 | 0.02 | 0.01 | 0.005 | 0.0025 |
| CX-1Cells/wellCV-1 | 1000 | 1000 | 1000 | 1000 | 1000 | 1000 | 1000 | 1000 |
| Final Conc. drug (ug/ml) | 3.2 | 1.6 | 0.8 | 0.4 | 0.2 | 0.1 | 0.05 | 0.025 |
| CV-1 | | | | | | | | |

3. Label the plates along the front, each row −1 compound.
4. Incubate the plates at 37° C. for 3 hrs.
   V. Rinsing
1. Prepare several bottles of CX-1 and CV-1 media.
2. Check for precipitate in first column of each plate and note.
3. Starting with the plates treated first, rinse several plates at a time by aspirating the treatments and refilling each well with 0.5-1.0 ml of the rinse media. Aspirate and refill 2 more times for a total of 3 rinses.
4. After aspirating the third rinse of each plate, use a repeating pipetter to add 750 ul of fresh media to each well.
5. Incubate the plates at 37° C. for 7-10 days. VI. Fixing and Staining
   VI. Fixing and Staining
   The CV-1 plates may be ready to be fixed before the CX-1 plates—check them after 6 days or so. When the CX-1 colonies are big enough to be easily seen by the naked eye, they are ready to be fixed. Record the date of fixation for each group.
1. Prepare 2% crystal violet in 70% ethanol and pour into a squeeze bottle.
2. Label the plates (use a pen which will not rinse off with the ethanol).
3. Aspirate or shake out the media and add stain to each well with the squeeze bottle.
4. Allow to “fix” for 10 minutes or longer and then shake out the stain and rinse the plates several times in a tub of water.
5. Drain upside down and allow the plates to air dry.
6. Attach a label to the side of each plate listing compounds, cell types and assay number.
   VII. IC₅₀ Determinations

The IC₅₀ is the concentration of drug, which causes colony number to be reduced to 50% of the negative control. It is determined by counting the colonies in each well. To be counted, a CV-1 colony must contain a minimum of 20 cells, and a CX-1 colony must contain a minimum of 50 cells. The number of colonies is plotted vs. the drug concentration and the IC₅₀ extrapolated from the graph.

### Example 1

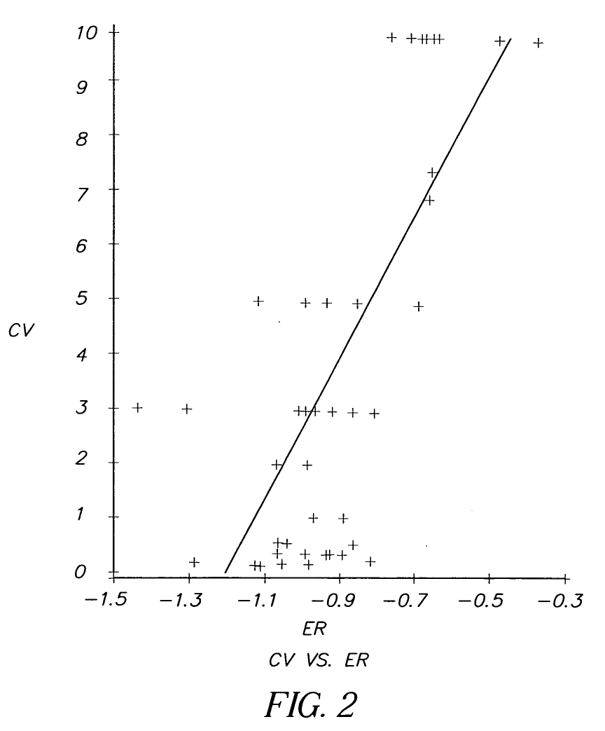

Evaluation of Dyes from Kodak advertisement (See Science April 30:1976; Scientific American May 1976). Table 1 shows the first results from the testing. It is seen that 3 of the dyes from the 18 dye series which appeared in the Kodak Advertisement, showed considerable anti-cancer activity relative to adriamycin, a widely used chemotherapeutic anti-cancer agent. This is also illustrated by FIG. 2.

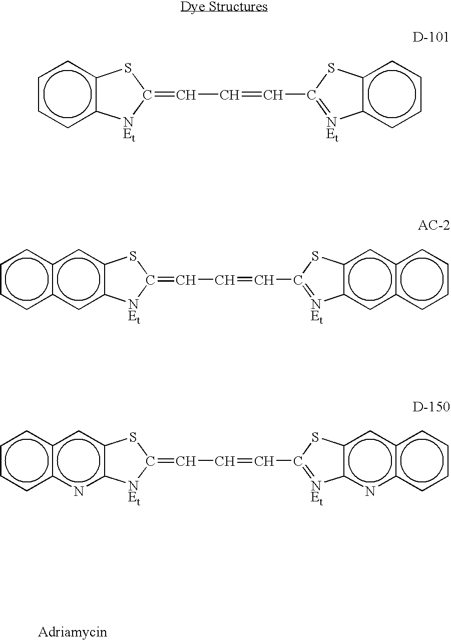

TABLE 1
Initial Screening Results

| Dye | ER(v) | Cell Line | IC₅₀ | Selectivity |
|---|---|---|---|---|
| D-101 | −1.06 | 1 | 0.50 | — |
| Kodak Ad | | 2 | 0.005 | 100 |
| Dye 6 | | 3 | 0.03 | 13.3 |
| | | 4 | 0.005 | 100 |
| | | 5 | <0.005 | >100 |
| | | 6 | .01 | 50 |
| | | 7 | .03 | 13.3 |
| | | 8 | <.005 | >100 |
| AC-2 | −.920 | 1 | 0.30 | — |
| Kodak Ad | | 2 | <.005 | >60 |
| Dye 7 | | 3 | <.005 | >60 |
| | | 4 | <.005 | >60 |
| | | 5 | <.005 | >60 |
| | | 6 | <.005 | >60 |
| | | 7 | <.005 | >60 |
| | | 8 | <.005 | >60 |
| D-150 | −.808 | 1 | 3.0 | — |
| Kodak Ad | | 2 | 0.03 | 100 |
| Dye 9 | | 3 | 0.03 | 100 |
| | | 4 | 0.03 | 100 |
| | | 5 | 0.01 | 300 |
| | | 6 | 0.02 | 150 |
| | | 7 | 0.10 | 30 |
| | | 8 | .01 | 300 |
| Adriamycin | −0.68 | 1 | 0.8 | — |
| | | 2 | 0.55 | 1.5 |
| | | 3 | 0.05 | 16 |
| | | 4 | 0.06 | 13 |
| | | 5 | 0.1 | 8 |
| | | 6 | 0.07 | 11 |
| | | 7 | 0.08 | 10 |
| | | 8 | 0.05 | 16 |
| D-132 | −1.01 | — | — | 428.57 |
| | −0.97 | | | |
| D-133 | −1.03 | — | — | 333.5 |
| | −0.99 | | | |
| D-148 | −1.15 | — | — | 250 |
| D-135 | −0.88 | — | — | 200 |
| | −0.84 | | | |
| D-104 | −0.96 | — | — | 200 |
| | −0.92 | | | |
| D-105 | −0.86 | — | — | 112.5 |
| | −0.82 | | | |
| D-100 | −0.9 | — | — | 100 |
| | −0.86 | | | |
| D-168 | −0.9 | — | — | 100.0 |
| D-112 | −1.1 | — | — | 900 |

Description of Cell Line
1. CV-1 Normal Monkey Kidney Epithelial (control)
2. CX-1 Human Colon Carcinoma
3. Human Breast Carcinoma
4. Human Pancreatic Carcinoma
5. Human Bladder Carcinoma
6. Human Lung Carcinoma
7. Human Squamous Cell Carcinoma
8. Human Adrenal Cortex Carcinoma

It is seen that the three cyanine dyes from Table 1 were effective anti-cancer agents at much lower concentrations and higher selectivity than Adriamycin. These three dyes were characterized as having electrochemical reduction potentials of −1.06V, −0.920V and −0.808V respectively. The dyes from this first series with electrochemical reduction potentials more negative than −1.20V had much lower selectivity kill ratios as they were toxic to both healthy and cancer cells. The dyes from this first series with electrochemical reduction potentials less negative than −0.808V had much lower selectively kill ratios and were relatively much less toxic to both healthy and cancer cells.

It was these initial results, which initiated a much broader investigation of the anti-cancer activity properties of many additional cyanine dyes.

### Example 2

From the screening of over 2000 cyanine dyes for anti-cancer activity, a number of cyanine dyes were discovered to have exceptionally strong anti-cancer actively with selectivities better than any anti-cancer agents reported to date. The anti-cancer results for dyes with selectivities equal to or greater than 60 are shown in Table 2.

It is seen that there are a wide variety of cyanine dye structures that show anti-cancer activity with selective kill ratios of 60 or greater.

Not all of the dyes evaluated had measured electrochemical reduction potentials. Of the 2000 dyes evaluated for anti-cancer activity, it was found that a number of dyes with electrochemical reduction potentials between —1.1 V and −0.8V showed selective kill ratios of 20 or less, possibly for reasons of solubility and steric factors. However, all of the dyes with selective kill ratios of 60 or greater that did have measured electrochemical reduction potentials, fit into a window of —1.1V to −0.8V. This strong correlation of exceptional selective anti-cancer kill ratios for many cyanine dyes indicates the electrochemical reduction potential of an anti-cancer agent is a very strong predictor of anti-cancer properties.

TABLE II

| Sample # | CX | CV | CV/CX |
|---|---|---|---|
| D-100 | 0.005 | 0.5 | 100 |
| D-101 | 0.005 | 0.5 | 100 |
| D-102 | <0.005 | 0.8 | 160 |
| D-103 | 0.01 | 5.0 | 500 |
| D-104 | 0.005 | 1.0 | 200 |
| D-105 | 0.008 | 0.9 | 112.5 |
| D-106 | 0.01 | 0.8 | 80 |
| D-107 | 0.05 | 3.0 | 60 |
| D-108 | 0.045 | 3.2 | 71.15 |
| D-109 | <0.005 | 0.3 | 60 |
| D-110 | <0.005 | 0.3 | 60 |
| D-111 | 0.03 | 8.0 | 266.7 |
| D-112 | 0.01 | 9.0 | 900 |
| D-113 | 0.009 | 0.5 | 56 |
| D-114 | 0.05 | 3.0 | 60 |
| D-115 | 0.09 | 5.0 | 56 |
| D-116 | 0.005 | 0.4 | 80 |
| D-117 | 0.1 | 7.0 | 70 |
| D-118 | 0.08 | 7.5 | 93.75 |
| D-119 | 0.05 | 3.0 | 60 |
| D-120 | 0.01 | 5.0 | 500 |
| D-121 | 0.01 | 0.8 | 80 |
| D-122 | <0.005 | 0.4 | 80 |
| D-123 | 0.005 | 0.4 | 80 |
| D-124 | 0.1 | 10.0 | 100 |
| D-125 | 0.035 | >3.2 | 91 |
| D-126 | 0.1 | >10 | 100 |
| D-127 | 0.005 | 0.3 | 60 |
| D-128 | 0.005 | 0.3 | 60 |
| D-129 | 0.008 | 0.7 | 87.5 |
| D-130 | 0.005 | 0.5 | 100 |
| D-131 | <0.005 | 0.3 | 60 |
| D-132 | 0.007 | 3.0 | 428.57 |
| D-133 | 0.009 | 3.0 | 333.5 |
| D-134 | 0.0025 | 0.2 | 80 |
| D-135 | <0.005 | 1.0 | 200 |
| D-136 | <0.005 | 0.3 | 60 |
| D-137 | 0.03 | 3.0 | 100 |
| D-138 | <0.005 | 0.3 | 60 |
| D-139 | 0.03 | 3.0 | 100 |
| D-140 | <0.005 | 0.3 | 60 |
| D-141 | <0.005 | 0.5 | 100 |
| D-142 | <0.005 | 0.5 | 100 |
| D-143 | 0.1 | 10 | 100 |
| D-144 | 0.03 | 3.0 | 100 |
| D-145 | <0.005 | 0.3 | 60 |
| D-146 | <0.005 | 0.3 | 60 |
| D-147 | 0.05 | 8.0 | 160 |
| D-148 | 0.04 | >10 | 250 |
| D-149 | 0.09 | 7.0 | 77.8 |
| D-150 | 0.03 | 3.0 | 100 |
| D-151 | 0.007 | 0.5 | 71.42 |
| D-152 | <0.005 | 0.3 | 60 |
| D-153 | 0.03 | 3.0 | 100 |
| D-154 | 0.05 | 8.0 | 160 |
| D-155 | 0.09 | 5.0 | 56 |
| D-156 | 0.05 | 3.0 | 60 |
| D-157 | 0.1 | 10 | 100 |
| D-158 | 0.03 | 3 | 100 |
| D-159 | 0.03 | 3 | 100 |
| D-160 | 0.05 | 5 | 100 |
| D-161 | 0.08 | 5 | 62.5 |
| D-162 | 3.0 | 0.50 | 60.0 |
| D-163 | 0.3 | <0.005 | >60.0 |
| D-164 | 0.90 | 0.008 | 112.5 |
| D-165 | 0.5 | 0.005 | 100.0 |
| D-165 | 0.5 | 0.005 | 100.0 |
| D-167 | 10.0 | 0.10 | 100.0 |
| D-168 | 3 | 0.03 | 100.0 |
| D-169 | 3 | 0.03 | 100.0 |
| D-170 | 5 | 0.05 | 100.0 |
| D-171 | 5 | 0.08 | 62.5 |

### Example 3

### Chain Length

In general, it appears that carbocyanine dyes (3 methine carbon chain) have higher selectivity than simple cyanine (1 methine carbon) or dicarbocyanine dyes (5 methine carbons) for direct analogs. This apparent correlation may also be complicated if light is not eliminated completely during the screening process since the chain length has a high degree of influence on a dye's light stability. (See Under certain conditions, cyanine dyes can generate singlet oxygen, G. W. Byers, S. Gross, P. M. Henrichs, Photochem. Photobiol., 23, 37 (1976).) Dicarbocyanines and longer chain dyes tend to decompose readily in aqueous solutions in the presence of light.

TABLE III
Effects of Methine Chain Length

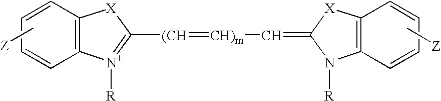

| | Z | X | m | CV | CX | CV/CX |
|---|---|---|---|---|---|---|
| D-165 | H | S | 1 | 0.5 | 0.005 | 100.0 |
| | H | S | 2 | 0.30 | 0.07 | 4.3 |
| | 5,6-Benzo | S | 0 | 0.10 | 0.005 | 20.0 |
| D-109 | 5,6-Benzo | S | 1 | 0.3 | <0.005 | >60.0 |
| D-162 | 5,6-Benzo | S | 2 | 3.0 | 0.50 | 60 |
| | 4,5-Benzo | 5 | 0 | 0.08 | 0.005 | 16.0 |
| | 4,5-Benzo | 5 | 1 | 2.0 | 0.03 | 10.0 |
| | H | 0 | 0 | >3.2 | >0.32 | 10.0 |
| | H | 0 | 1 | 3.0 | 0.10 | 30.0 |

### Example 4

### Nitrogen Substituents

For the dyes listed in Tables IV and V, methyl or ethyl substitution on both nitrogens generally results in the highest selectivity. However, it is possible to place certain substituents other than methyl or ethyl on one of the nitrogens and retain high selectivity, although placing that substituent on both nitrogens can greatly decrease activity/selectivity. For instance, the N-2-hydroxyethyl, N′-ethyl dye (D-125, Table IV) has high selectivity and activity when compared to the N,N′-di(2-hydroxyethyl) dye. (See the section on QSAR for art analysis of this effect.)

TABLE IV
Nitrogen Substitution on the Thiacarbocyanine Chronophore

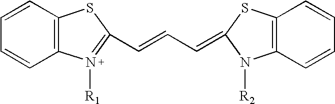

| Sample ID | R1 | R2 | CV | CX | CV/CX |
|---|---|---|---|---|---|
| D-101 | Et | Et | 0.5 | <0.005 | >100.0 |
| D-100 | Me | Me | 0.5 | 0.005 | 100.0 |
| D-125 | CCOH | Et | >3.2 | 0.035 | >91.4 |
| | CCSMe | CCSMe | 0.18 | 0.015 | 12.0 |
| | CCC(SEt)2 | CCC(SEt)2 | 1.3 | >0.32 | <4.1 |
| | CCOH | CCOH | >3.2 | >0.32 | 10.0 |
| | CC(═O)morpholino | H | >3.2 | >0.32 | 10.0 |

TABLE V
Nitrogen Substitution on the, Oxathiacarbocyanine Chromophore

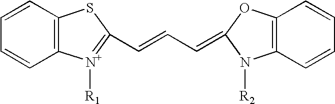

| ID | R1 | R2 | CV | CX | CV/CX |
|---|---|---|---|---|---|
| D-104 | Et | Et | 1.0 | 0.005 | 200.0 |
| D-124 | CCN | Et | 10.0 | 01 | 100.0 |
| D-122 | iPr | Et | 0.4 | <0.005 | >80.0 |
| | CCC═O | Et | 5.0 | 0.1 | 50.0 |
| | Et | CC(F)3 | 5.0 | 0.1 | 50.0 |
| | CCSO2CC | Et | >10.0 | 0.3 | >33.3 |
| | Cc(═0)N(Et)2 | Et | >10.0 | 0.3 | >33.3 |
| | CC(═O)Ph-2, 4-OH | Et | >10.0 | 0.3 | >33.3 |
| | CCC═NNSO2Ph-4C | Et | 10.0 | 0.3 | 33.3 |
| | CC(Br)═C | Et | 1.0 | 0.03 | 33.3 |
| | CCNSO2Ph-4C | Et | 8.0 | 0.3 | 26.7 |
| | CCC(═0)N(iPr)Ph | Et | 0.8 | 0.03 | 26.7 |
| | CC(═O)N | Et | >10.0 | 1.0 | >10.0 |
| | CC(═O)Ph | Et | 3.0 | 0.3 | 10.0 |
| | Et | C(C═O)Ph | 3.0 | 0.8 | 3.7 |
| | CC(═O)morpholino | Et | >10.0 | >1.0 | 10.0 |
| | CCC═NNC(═S)N | Et | >10.0 | >1.0 | 10.0 |
| | CC(═O)Ph-3, 4-OH | Et | >10.0 | >1.0 | 10.0 |

### Example 5

### Chain Substitution

Chain substituents decrease selectivity, although not necessarily activity. The chain substituent would be expected to affect steric, electronic and hydrophobicity factors. It could also change the conformation of the chain from transoid to cisoid. Dyes with meso thio-alkyl substituents in some cases retain good, although lower selectivity. For instance, dye D-142 (Table VII) a 9-ethylthio-oxathiacarbocyanine, has a CV/CX ratio of greater than 100, whereas the corresponding unsubstituted dye (D-104) has a selectivity of 200. However, dye D-142 has lower IC₅₀ values (higher activity) in both the CX and CV cell lines.

TABLE VI
Chain Substitution on the Thiacarbocyanine Chromophore

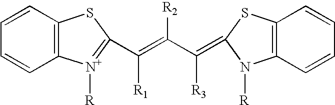

| ID | R^{a} | R1 | R2 | R3 | CV | CX | CV/CX | |
|---|---|---|---|---|---|---|---|---|
| D-100 | M | H | H | H | 0.5 | 0.005 | 100.0 | |
| D-101 | E | H | H | H | 0.5 | 0.005 | 100.0 | |
| | E/M | H | SMe | H | 0.3 | 0.008 | 37.5 | |
| | M | H | SMe | H | 0.4 | 0.015 | 26.6 | |
| | E | H | Set | H | 0.6 | 0.04 | 15.0 | |
| | M | H | Et | H | 0.07 | 0.005 | 14.0 | |
| | E | H | SPhNO2 | H | 1.8 | 0.16 | 11.25 | |
| | E | H | C(═S)SC | H | >3.2 | 0.32 | >10.0 | |
| | E | H | Et | H | 0.05 | 0.005 | 10.0 | |
| | E | H | 2Thienyl | H | 1.6 | 0.16 | 10.0 | |
| | E/M | H | SPh | H | 0.7 | 0.07 | 10.0 | |
| | E | H | C(═S)S— | H | 2.8 | >0.32 | <8.7 | |
| | E | H | Me | H | 0.04 | 0.005 | 8.0 | |
| | M | H | SPh | H | 0.5 | 0.065 | 7.69 | |
| | E | H | SPhOMe | H | 0.35 | 0.05 | 7.0 | d5 |
| | M | H | Me | H | 0.1 | 0.02 | 5.0 | |
| | E | H | SPhMe | H | 0.25 | 0.07 | 3.57 | |
| | M | Me | H | Me | 0.5 | 0.2 | 2.5 | |
| | M | Me | H | Me | 0.5 | 0.2 | 2.5 | |
| | E | H | SMe | H | 0.3 | 0.24 | 1.25 | |
| | M | CHO | H | H | >3.2 | >0.32 | 10.0 | |

^{a}M = methyl, E = Ethyl.

TABLE VII
Chain substitution on the Oxathiacarbocyanine Chromophore

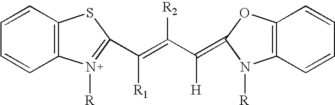

| ID | R^{a} | R1 | R2 | CV | CX | CV/CX |
|---|---|---|---|---|---|---|
| D-104 | E | H | H | 1.0 | 0.005 | 200.0 |
| D-142 | E | H | SEt | 0.5 | <0.005 | >100.0 |
| D-139 | E | Ph | H | 3.0 | 0.03 | 100.0 |
| D-137 | E | H | SMe | 3.0 | 0.03 | 100.0 |
| D-127 | M/E | H | SEt | 0.3 | 0.005 | 60.0 |
| | E | H | Et | 1.0 | 0.03 | 33.3 |
| | E | H | CSC(═O)C | 1.0 | 0.03 | 33.3 |
| | E | H | Me | 1.0 | 0.05 | 20.0 |
| | E | OAc | H | 10.0 | 1.0 | 10.0 |
| | E | OH | H | 10.0 | 1.0 | 10.0 |
| | E/M | H | 9-Phenanthyl | 1.0 | 0.3 | 3.3 |
| | M/E | SPh | H | 3.0 | 1.0 | 3.0 |
| | M/E | H | SPh | 0.3 | 0.1 | 3.0 |

^{a}M = methyl, E = Ethyl.

TABLE VIII
Chain Substitution on the Naptholl₁21thiacarbocyanine Chromophore

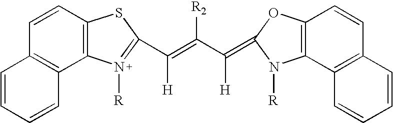

| | | | | | |
|---|---|---|---|---|---|
| ID | R^{a} | R2 | CV | CX | CV/CX |

| | | | | |
|---|---|---|---|---|
| M | Ph | 0.3 | 0.03 | 10.0 |
| E | H | 2.0 | 0.3 | 6.7 |
| MH | Me | 0.2 | 0.03 | 6.7 |
| E | CSC(═S)C | 0.8 | 0.12 | 6.7 |
| E | SMe | 0.2 | 0.04 | 5.0 |
| E | Et | 0.3 | 0.06 | 5.0 |
| M | Et | 0.4 | 0.08 | 5.0 |
| E | Me | 0.1 | 0.03 | 3.33 |
| E | Me | 0.1 | 0.05 | 2.0 |
| E | SEt | 0.05 | 0.04 | 1.25 |

^{a}M = methyl, E = Ethyl.

### Example 6

### Back-Ring Substituents (Tables IX-XIII)

For certain dyes, back-ring substitution can apparently increase selectivity. This is particularly true for the selenathiacarbocyanines listed in Table X. The unsubstituted dye (D-110) has a CV/CX value of >60 and the 5-methoxy (D-134) and 5-chloro (D-135) dyes have CV/CX ratios of 80 and >200, respectively.

TABLE IX
Back-ring Substitution on the Thiacarbocyanine Chromophore

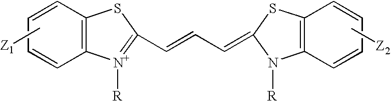

| ID | R^{a} | Z1 | Z2 | CV | CX | CV/CX |
|---|---|---|---|---|---|---|
| D-164 | Et | 6-Cl | 6-Cl | 0.90 | 0.008 | 112.5 |
| D-100 | Me | H | H | 0.5 | 0.005 | 100.0 |
| D-165 | Et | H | H | 0.5 | 0.005 | 100.0 |
| D-146 | M/E | 4,5,6,7-H | H | 0.3 | 0.004 | 75.0 |
| | Et | 5-MeO | 5-MeO | 0.20 | 0.005 | 40.0 |
| | ET | 5-Cl | 5-Cl | 1.6 | 0.04 | 40.0 |
| | Et | 5-MeO | H | 0.2 | 0.005 | 40.0 |
| | Et | 5-Cl | H | 0.8 | 0.02 | 40.0 |
| | Et | 5-Br | 5-Br | 1.4 | 0.04 | 35.0 |
| | Et | 5,6-MeO | H | 0.4 | 0.02 | 20.0 |
| | M/E | 6-CN | H | 3.2 | 0.16 | 20.0 |
| | Et | 5-Cl | 5-Cl | 5.0 | 0.30 | 16.7 |
| | Et | 5-MeO | 5-Me | 0.2 | 0.015 | 13.3 |
| | M/E | 5-MeCO2- | H | 0.4 | 0.035 | 11.4 |
| | Et | 5,5-OH | H | 3.2 | 0.32 | 10.0 |
| | Et | 6-MeO | 6-MeO | 0.09 | 0.01 | 9.0 |
| | Et | 5-Cl, 7-N(Me)2 | H | 0.2 | 0.03 | 6.7 |
| | Et | 7-MeO | 7-MeO | 0.025 | 0.0050 | 5.0 |
| | Et | 5-Ph | 5-Ph, | 0.1 | 0.08 | 1.25 |
| | Me | 6-SO2NMe | 6-SO2NMe | >3.2 | >0.32 | 10.0 |
| | Me | 4,5,6,7-F | 4,5,6,7-F | >3.2 | >0.32 | 10.0 |

^{a}M = methyl, E = Ethyl.

TABLE X
Back-ring Substitution on the Selenathiacarbocyanine Chromophore

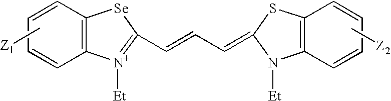

| ID | Z1 | Z2 | CV | CX | CV/CX |
|---|---|---|---|---|---|
| D-135 | 5-Cl | H | 1.00 | <0.005 | >200.0 |
| D-134 | 5-MeO | H | 0.20 | 0.0025 | 80.0 |
| D-110 | H | H | 0.30 | <0.005 | >60.0 |
| | H | 5,6-OH | 10.00 | 0.300 | 33.3 |

^{a}M = methyl, E = Ethyl.

TABLE XI
Back-ring Substitution on the Naphthothiacarbocyanine Chromophore

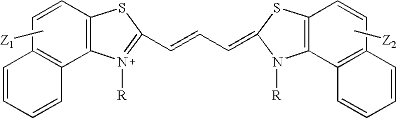

| ID | R^{a} | Z1 | Z2 | CV | CX | CV/CX |
|---|---|---|---|---|---|---|
| | M | 8-MeO | 8-MeO | 0.8 | 0.02 | 40.0 |
| | M | 7-MeO | 7-MeO | 3.2 | 0.09 | 35.5 |
| | M | 6-MeO | 6-MeO | 0.8 | 0.08 | 10.0 |
| | M | H | H | 0.05 | 0.005 | 10.0 |
| | E | 4,5-H | 4,5-H | 0.05 | 0.006 | 8.33 |
| | E | H | H | 2.0 | 0.3 | 6.7 |
| | M | 4,5-H | 4,5-H | 0.80 | 0.20 | 4.0 |
| | M | 4,5-H, 5-ME | 4,5-H, 5-ME | 0.10 | 0.08 | 1.25 |
| | M | 4,5-H, 8-MeO | 4,5-H, 8-MeO | 0.05 | 0.04 | 1.25 |
| | M | 4,5-H, 6-MeO | 4,5-H, 6-MeO | 0.015 | 0.02 | 0.75 |

TABLE XII
Back-ring Substitution on the Oxathiacarbocyanine Chromophore

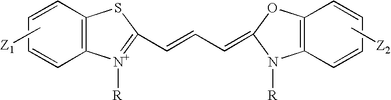

| ID | R^{a} | Z1 | Z2 | CV | CV | CV/CX |
|---|---|---|---|---|---|---|
| D-104 | E | H | H | 1.0 | 0.005 | 200.0 |
| D-106 | E | 4-Ph | H | 0.8 | 0.01 | 80.0 |
| D-138 | E/M | H | 5-SMe | 0.3 | <0.005 | >60.0 |
| D-136 | E | H | 5-MeO | 0.3 | <0.005 | >60.0 |
| D-131 | E | H | 5-Ph | 0.3 | <0.005 | >60.0′ |
| D-163 | E | 5,6-OCO— | H | 0.3 | <0.005 | >60.0 |
| D-128 | E | 5-OH | H | 0.3 | 0.005 | 60.0 |
| | Et | H | 5-Cl | 1.5 | 0.055 | 27.3 |
| | M/E | 6-CN | H | 8.0 | 0.3 | 26.7 |
| | E | 5,6-Me | H | 0.1 | <0.005 | >20.0 |
| | E | H | 5,6-Me | 0.1 | <0.005 | >20.0 |
| | E | H | 5-NHSO2Ph | 5.0 | 0.3 | 16.7 |
| | E | 5-CN | H | >3.2 | 0.24 | >13.3 |
| | E | 5,6-MeO | H | 0.5 | 0.04 | 12.5 |
| | E | H | 5-Cl, 6-Me | 0.4 | 0.035 | 11.4 |
| | M/E | H | H | 0.06 | 0.006 | 10.0 |
| | E | 5,6-OH | H | 8.0 | 1.0 | 8.0 |
| | E | H | 5-Me | 0.8 | 0.2 | 4.0 |
| | E | H | 4-OH | 3.0 | 7.0 | 0.43 |
| | E/M | 6-N03 | 5-Cl, 6-N03 | >10. | >1.0 | 10.0 |

^{a}M = methyl, E = Ethyl.

TABLE XIII
Back-ring Substitution on the Oxathiacarbocyanine Chromophore

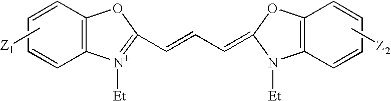

| ID | Z1 | Z2 | CV | CX | CV/CX |
|---|---|---|---|---|---|
| | H | H | 3.0 | 0.10 | 30.0 |
| | 5-Cl, 6-Me | 5-Cl, 6-Me | 2.0 | 0.16 | 12.5 |
| | 5,7-Cl, 6-Me | 5,6-Cl, 7-Me | >3.2 | 0.32 | >10.0 |
| | 5-MeO | 5-MeO | 1.6 | 0.31 | 5.16 |
| | 5,6-OCO— | 5,6-OCO— | 0.4 | 0.08 | 5.0 |
| | 7-Ph | 7-Ph | 0.8 | 0.16 | 5.0 |
| | 5-CF3 | 5-CF3 | >3.2 | >.32 | 10.0 |
| | 5-CO2Et | 5-CO2Et | >3.2 | >.32 | 10.0 |
| | 5-F | 5-F | >3.2 | >.32 | 10.0 |

### Example 7

### Dyes with Potential Hydrogen Bonding Substituents (Table XIV)

If intercalation is important, then hydrogen-bonding substituents should increase selectivity, as described in H. W. Zimmerman, Angew. Chem. Int. Ed., 25, 115-130 (1986). Table XIV seems to indicate that back-ring hydroxy substituents do not increase selectivity. However, the one example of a 6-amino substituted dye did show a significant increase in selectivity (CV/CX=40) over the parent dye CV/CX=13).

TABLE XIV
Dyes with Potential Hydrogen Bonding Substituents

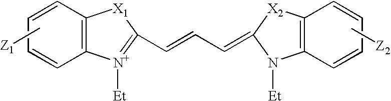

| ID | X1 | X2 | Z1 | Z2 | CV | CX | CV/CX |
|---|---|---|---|---|---|---|---|
| D-165 | S | S | H | H | 0.5 | 0.005 | 100.0 |
| | S | S | 5,6-OH | H | 3.2 | 0.32 | 10.0 |
| | Te | Te | H | H | 5.0 | 0.30 | 16.7 |
| | Te | Te | 5-OH | 5-OH | >3.2 | >3.20 | |
| D-104 | S | O | H | H | 1.0 | 0.005 | 200.0 |
| D-128 | S | O | 5-OH | H | .03 | 0.005 | 60.0 |
| | S | O | 5,6-OH | H | 8.0 | 1.0 | 8.0 |
| D-110 | Se | S | H | H | 0.30 | <0.005 | >60.0 |
| | Se | S | H | 5,6-OH | 10.0 | 0.30 | 33.3 |

Amino Substituents

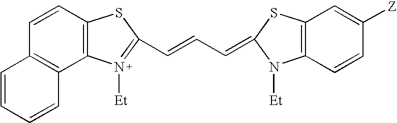

| | | | | |
|---|---|---|---|---|
| ID | Z | CV | CX | CV/CX |

| | | | |
|---|---|---|---|
| H | 4.0 | 0.3 | 13.3 |
| NH2 | 4.0 | 0.10 | 40.0 |

### Example 8

### Symmetrical Dyes

Some symmetrical dyes containing various heterocycles are listed in Table XV. Indole has the highest selectivity but in vivo tests indicate that it also has high toxicity. Benzothiazole is by far the next best.

TABLE XV
Symmetrical Dyes with Different Heterocycles

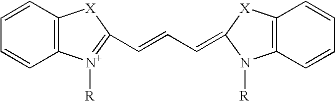

| ID | B | R^{a} | X | CV | CX | CV/CX |
|---|---|---|---|---|---|---|
| | B-400 | E | 0 | 3.0 | 0.10 | 30.0 |
| D-101 | B-335 | E | S | 0.5 | 0.005 | 100.0 |
| | B-405 | M | Se | 0.06 | 0.008 | 7.5 |
| | B19682 | M | Te | 5.0 | 0.3 | 16.7 |
| D-103 | H-92 | E | C(CH₃)₂ | 5.0 | 0.010 | 500.0 |
| | B-301 | E | —C═C— | 0.50 | 0.040 | 12.5 |

^{a}M = methyl, E = Ethyl.

### Example 9

### Unsymmetrical Dyes

Table XVI lists dyes containing the benzothiazole nucleus and various heterocycles as the other nucleus. When the second nucleus is benzoxazole (D-104, CV/CX=200) or isoindole (D-132, CV/CX=430), the dyes have significantly higher selectivity than the symmetrical thiacarbocyanine (D-165, CV/CX=100).

TABLE XVI
Unsymmetrical Dyes Heterocycles

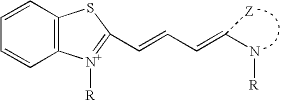

| ID | Heterocycle | R^{a} | CV | CX | CV/CX |
|---|---|---|---|---|---|
| D-165 | benzothiazole | E | 0.5 | 0.005 | 100.0 |
| D-104 | benzoxazole | E | 1.0 | 0.005 | 200.0 |
| D-152 | benzotellurazole | E/M | 0.3 | <0.005 | >60.0 |
| | naphtho/2,3/thiazole | E | 0.4 | 0.007 | 57.0 |
| D-110 | benzoselenazole | E | 0.3 | <0.005 | >60.0 |
| | benzimidazole(5,6-Cl) | E | 0.2 | 0.260 | 0.8 |
| | naphtho/1,2/thiazole | M | <0.05 | <0.005 | 10.0 |
| | naphtho/1,2/thiazole | E/M | 4.0 | 0.300 | 13.3 |
| D-123 | 5-Ph-oxazole | E | 0.40 | 0.005 | 80.0 |
| | 2-oxazoline | E | 3.0 | 0.30 | 10.0 |
| | naphtho/2,1/thiaole | M | 0.04 | <0.005 | >10.0 |
| | (4,5-dihydro) | | | | |
| | 5-Ph-thiazole | E | 0.50 | 0.03 | 16.7 |
| | 3H-indole (3,3-Me) | E | 0.05 | 0.16 | 0.3 |
| | 4-quinoline | E/M | 0.40 | 0.01 | 40.0 |
| | 2-quinoline | M/E | 0.10 | <0.005 | >20.0 |
| | benzothiazole(4,5,6,7-H) | E/M | 0.30 | <0.005 | >60.0 |
| | 2-pyridine | E/M | 4.0 | 0.08 | 50.0 |
| | naphtho/1,2/tellurazole | E/M | 0.3 | 0.10 | 3.0 |
| D-132 | 1/3H-isoindole | E/M | 3.0 | 0.007 | 428.6 |
| | 4-thieno/2,3-b/pyridine | E | 0.2 | 0.04 | 5.0 |
| D-129 | 6-Phenanthridine | E/M | 0.7 | 0.008 | 87.5 |

^{a}M = methyl, E = Ethyl.

### Example 10

### Benzotellurazole Dyes (Table XVII)

Table XVII lists some benzotellurazole dye derivatives. Two of the dyes listed (D-151 and D-152) have high CV/CX values (71 and >60, respectively).

TABLE XVII
Benzotellurazole Derivatives

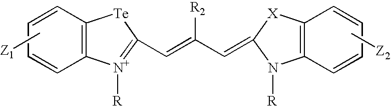

| ID | R^{a} | X | Z1 | R2 | Z2 | CV | CX | CV/CX |
|---|---|---|---|---|---|---|---|---|
| D-151 | M/E | S | H | H | 5,6-Benzo | 0.5 | 0.007 | 71.4 |
| D-152 | M/E | S | H | H | H | 0.3 | <0.005 | >60.0 |
| | E | S | 5-Me | Me | 5-MeO | 1.0 | 0.03 | 33.3 |
| | E | S | 5-Me | Ph | H | 9.0 | 0.3 | 30.0 |
| | E | S | 5-MeO | Ph | H | 8.0 | 0.3 | 26.6 |
| | E | S | 5-Me | Me | H | 0.8 | 0.03 | 26.6 |
| | E | S | 5-Me | Me | 5-Cl | 1.0 | 0.10 | 10.0 |
| | M/E | S | 5,6-MeO | H | H | 1.0 | 0.5 | 2.0 |
| | M | Te | H | H | H | 5.0 | 0.30 | 16.7 |
| | E | Te | 5-Me | H | 5-Me | 0.4 | 0.052 | 7.7 |
| | M | Te | 5-OH | H | 5-OH | >3.2 | >0.42 | 7.6 |

^{a}M = methyl, E = Ethyl.

### Example 11

### Complex Cyanines (Table XVIII)

Cyanine dyes with three heterocycles are called 'complex cyanines. In general, these dyes were less toxic to both normal and transformed cells than the regular cyanines; however, many of the dyes showed high selectivity (e.g., dye D-111 has a CV/CX value of 270).

Because of their structure variation, it is difficult to tabulate them. A few are in Table XVIII.

TABLE XVIII
Complex Cyanines

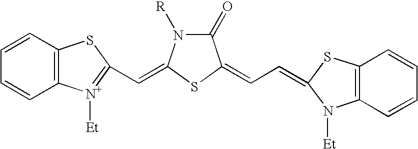

| ID | R | CV | CX | CV/CX |
|---|---|---|---|---|
| D-111 | Me | 8.0 | 0.03 | 266.7 |
| D-167 | CCCO₂— | 10.0 | 0.10 | 100.0 |
| | Et | 1.6 | 0.07 | 22.8 |

### Example 12

### Redox Properties

It appears that there is some linear correlation between the reduction potential (Er) and toxicity. FIGS. 3 and 4 are plots of reduction potential (Er) versus CX (r=0.71) and CV (r=0.72), respectively. As reduction potential (Er) becomes more negative, the IC₅₀ values decrease (toxicity increases). Reduction potential (Er) is plotted versus CX/CV in FIG. 5. Since chain substitution lowers selectivity, this could affect the correlation, and dyes with chain substituents are marked on the plot. There are still a number of dyes that have the ‘correct reduction potential’ but have low selectivity. This should not be surprising since even if the reduction potential (Er) was critical many other factors may also influence selectivity.

TABLE XIX
Redox Properties

| ID | Set | Er^{a} | Eox^{a} | CX | CV | CV/CX |
|---|---|---|---|---|---|---|
| | 0 | −0.390 | 1.410 | 1.000 | 10.000 | 10.000 |
| | 0 | −0.490 | 1.305 | 1.000 | 10.000 | 10.000 |
| | 0 | −0.646 | 1.211 | 1.000 | 10.000 | 10.000 |
| | 0 | −0.650 | 1.515 | 1.000 | 10.000 | 10.000 |
| | 0 | −0.661 | 1.490 | 1.000 | 10.000 | 10.000 |
| | 4 | −0.661 | 1.490 | 1.000 | 10.000 | 10.000 |
| | 0 | −0.663 | 1.430 | 1.000 | 10.000 | 10.000 |
| | 0 | −0.666 | 1.621 | 0.500 | 7.500 | 15.000 |
| | 2 | −0.670 | 0.786 | 1.000 | 7.000 | 7.000 |
| | 0 | −0.689 | 1.184 | 1.000 | 10.000 | 10.000 |
| | 2 | −0.695 | 1.417 | 1.000 | 5.000 | 5.000 |
| | 2 | −0.697 | 1.140 | 1.000 | 10.000 | 10.000 |
| | 2 | −0.725 | 1.441 | 1.000 | 10.000 | 10.000 |
| | 2 | −0.728 | 1.048 | 1.000 | 10.000 | 10.000 |
| | 0 | −0.780 | 1.096 | 1.000 | 10.000 | 10.000 |
| | 3 | −0.780 | 1.096 | 1.000 | 10.000 | 10.000 |
| | 1 | −0.808 | 1.145 | 0.020 | 0.200 | 10.000 |
| D-150 | 0 | −0.808 | 1.145 | 0.030 | 3.000 | 100.000 |
| | 2 | −0.855 | 0.680 | 0.010 | 0.500 | 50.000 |
| | 3 | 0.860 | 0.550 | 1.000 | 5.000 | 5.000′ |
| | 4 | −0.865 | 0.330 | 1.000 | 3.000 | 3.000 |
| | 4 | −0.884 | 0.513 | 0.300 | 1.000 | 3.333 |
| | 1 | −0.885 | 0.630 | 0.070 | 0.300 | 4.286 |
| D-109 | 1 | −0.920 | 1.015 | 0.004 | 0.300 | 75.000 |
| D-160 | 0 | −0.920 | 1.015 | 0.004 | 0.300 | 75.000 |
| | 2 | −0.921 | 0.938 | 0.200 | 3.000 | 15.000 |
| | 2 | −0.928 | 0.720 | 0.100 | 0.300 | 3.000 |
| | 1 | −0.929 | 0.613 | 0.070 | 0.300 | 4.286 |
| | 0 | −0.943 | 0.976 | 0.300 | 5.000 | 16.667 |
| | 2 | −0.965 | 0.590 | 0.200 | 1.000 | 5.000 |
| | 2 | −0.968 | 0.412 | 0.500 | 3.000 | 6.000 |
| | 2 | −0.975 | 0.570 | 0.005 | 0.100 | 20.000 |
| | 2 | −0.985 | 0.520 | 0.300 | 0.300 | 1.000 |
| | 2 | −0.985 | 0.500 | 0.500 | 2.000 | 4.000 |
| | 2 | −0.994 | 0.419 | 0.500 | 3.000 | 6.000 |
| D-103 | 3 | −1.000 | 1.088 | 0.010 | 5.000 | 500.000 |
| | 0 | −1.013 | 0.976 | 0.300 | 3.000 | 10.000 |
| | 3 | −1.035 | 0.757 | 0.040 | 0.500 | 12.500 |
| | 3 | −1.048 | 0.902 | 0.080 | 0.100 | 1.250 |
| D-101 | 1 | −1.060 | 0.902 | 0.005 | 0.500 | 100.000 |
| D-101 | 5 | −1.060 | 0.902 | 0.004 | 0.300 | 75.000 |
| | 1 | −1.068 | 0.770 | 0.300 | 2.000 | 6.667 |
| | 0 | −1.068 | 0.770 | 0.300 | 2.000 | 6.667 |
| | 1 | −1.107 | 0.901 | 0.005 | 0.050 | 10.000 |
| | 1 | −1.120 | 0.902 | 0.005 | 0.070 | 14.000 |
| | 0 | −1.128 | 1.113 | 0.750 | 5.000 | 6.667 |
| | 0 | −1.280 | 0.634 | 0.100 | 0.100 | 1.000 |
| | 0 | −1.310 | 1.020 | 0.100′ | 3.000 | 30.000 |
| | 1 | −1.445 | 1.370 | 0.100 | 3.000 | 30.000 |

^{a}vs. Ag/AgCl.
CV vs. Er

### Example 13

### Quantitative Structure/Activity Relationships (OSAR)

Determination of Quantitative Structure/Activity Relationships (the Hansch approach) is one method used to predict active structures and gain insight into mechanisms of action. The assumption is that the effect of substituents on the activity of a drug can be separated into several independent factors. For instance, in this case, the activity of the dye might be a function of hydrophobicity effects (transport of the dye into the cell), steric effects, electronic effects and other factors (e.g., reduction potential, DNA binding constant, etc.). Ideally, by assigning a quantitative value for each of these parameters for a wide range of substituents, one could determine which factors are most important and derive an equation that would predict activity/selectivity.

Octanol/water partition coefficients (log P) are usually used as a model of drug transport between water and the biophase, as disclosed in A. Leo, C. Hansch, Substituent Constants—for Correlation Analysis in Chemistry. and' Biology, Wiley, New York, 1979 and A. Leo, C. Hansch, D. Elkins, Chem. Rev., 6, 525 (1971), both incorporated herein by reference. Often partition coefficients can be measured simply by shaking the compound with a mixture of water and octanol and determining the concentration of the solute in either solvent. P is equal to the concentration of the solute in octanol divided by its concentration in water. In general, it is possible to measure log P values ranging from −4.0 to 6.0. The situation is also more complicated when the species is charged because of the effects of ion pairing.

Several cationic cyanine dyes, including 3,3′-diethylthiacarbocyanine iodide, were examined and were found to partition completely into the octanol phase. Consequently, this suggests that for most cationic dyes, it will not be possible to determine a partition coefficient directly.

Perhaps the easiest approach is to examine only the effect of substituents on hydrophobicity. In this case, it is possible to use substituent hydrophobicity parameters (π). A positive value for π means that, relative to H, the substituent favors the octanol phase. Values for various substituents have been tabulated or they can be estimated by using the Medchem program (a recent version of the Medchem program is available from BioByte Corp., 204 W. 4^{th} St., #204, Claremont, Calif.

Many parameters have been used to quantify the steric effects of substituents including Taft parameters (Es) (see R. W. Taft, J. Amer.-Chem. Soc, 77744, 3120 (1952)) and Verloop-Hoogenstraaten multidimensional steric parameters (L, B1, B2, B3, B4) (see A. Verloop, W. Hoogenstraaten J. Tipker in Drug, Design, E. J. “Ariens, Ed.; Academic Press, New York, 1976, vol VII, 165. A. Verloop, ‘ In Pesticide Chemistry’ A Human Welfare and the Environment, J. Miyamoto, P. C.” Keamey, Ed., Pergamon Press, New York, 1982, 339). Molar refractivity is often used as a steric parameter because it is related to molar volume (it is also related to the polarizability of the molecule}. The Medchem program can be used to estimate a compound's molar refractivity.

Electronic effects are most commonly correlated with the Hammett a values of the substituents, as described in H. H. Jaffe, Chem. Rev., 53, 191, (1953.) incorporated herein by reference, although other parameters have also been used extensively, such as Swain and Lupton F and R values, described in C. G. Swain E. C. Lupton, J. Amer. Chem. Soc., 90, 4328 (1968), incorporated herein by reference.

Additional factors which might be considered important (e.g., redox potentials) generally need to be measured for individual compounds.

Table XX lists the substituents, the sum of their hydrophobicity parameters:(π), and the sum of their molar refractivity values (MR), which will be used as a steric parameter. The MR values have been scaled by multiplying them by 0.1 to make them comparable in size to the hydrophobicity parameters. Where possible, the parameters were taken from the literature; however, many of the substituents are fairly exotic and it was necessary to estimate their value using the Medchem program, in which the P value of a substituent R was estimated by calculating the logP value for the parent compound (RH) and subtracting the logP value for hydrogen (0.45), i.e., P(R)=logP(R—H)— logP (H—H) and in which the MR Value of the substituent (R) was taken as equal to that calculated for the parent (RH), introducing considerable error into the analysis. The octanol/water partition coefficient (P) of a compound is the ratio of the amount of material that dissolves in the octanol phase divided by the concentration in the aqueous phase at equilibrium. LogP is often used to describe the relative tendency of a molecule to favor an oil (octanol) or water phase (see Leo and Hansch, “Substituent Constants for Correlation Analysis in Chemistry and Biology,” Wiley, New York, 1979, and in Leo, Hansch, and Elkins, Chem. Rev., 6, 525, (1971)). It is a measure of how hydrophobic or hydrophilic the molecule is.

By using regression analysis, a correlation table can be generated between these parameters and the dye's selectivity (CX/CV) and activities. For cases where the dye was inactive, that is, the IC₅₀ values were greater than the upper limit, the CV/CX ratio was given a value of 1. One common problem when applying this approach is that often the substituent parameters will be colinear. For this data set, the correlation coefficient between the π and MR values is 0.67.

TABLE XX
Nitrogen Substituent Parameters for the Thiacarbocyanine Chromophore

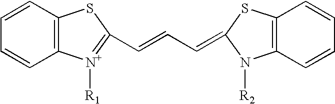

| ID | R1 | R2 | CV | CX | CV/CX | π | MR |
|---|---|---|---|---|---|---|---|
| D-100 | Et | Et | 0.5 | <0.005 | 100.0 | 2.04 | 2.06 |
| D-100 | Me | Me | 0.5 | 0.005 | 100.0 | 1.12 | 1.13 |
| D-125 | CCOH | Et | >3.2 | 0.035 | 91.4 | 0.61 | 2.28 |
| | CCSMe | CCSMe | 0.18 | 0.015 | 12.0 | 1.84 | 4.75 |
| | CCC(SEt)2 | CCC(SEt)2 | 1.3 | >0.32 | 4.1 | 4.81 | 10.07 |
| | CCOH | CCOH | >3.2 | >0.32 | 1.0 | −1.38 | 2.51 |
| | CC(═0)morpholino | H | >3.2 | >0.32 | 1.0 | −0.54 | 4.83 |

Correlation Coefficients

| | CX | CV | CV/CX | π | MR |
|---|---|---|---|---|---|
| CX | 1.00000 | 0.59180 | −0.79562 | −0.12656 | 0.57617 |
| CV | | 1.00000 | −0.30158 | −0.62539 | −0.05146 |
| CV/CX | | | 1.00000 | 0.05980 | −0.65047 |
| W | | | | 1.00000 | 0.66729 |
| | | | | | 1.00000 |

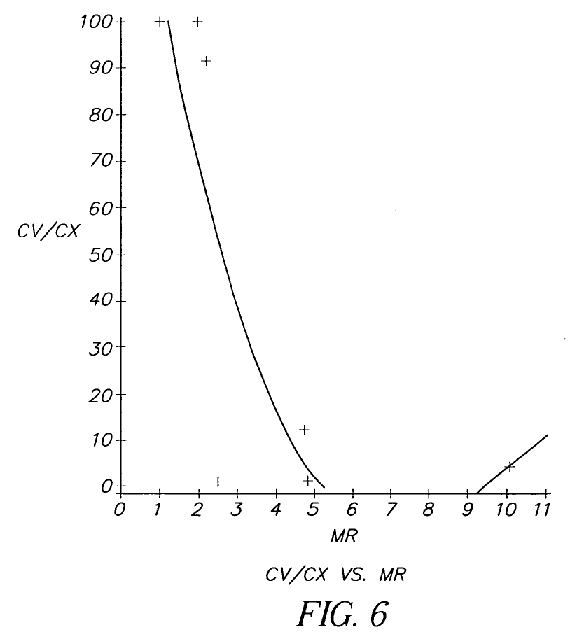

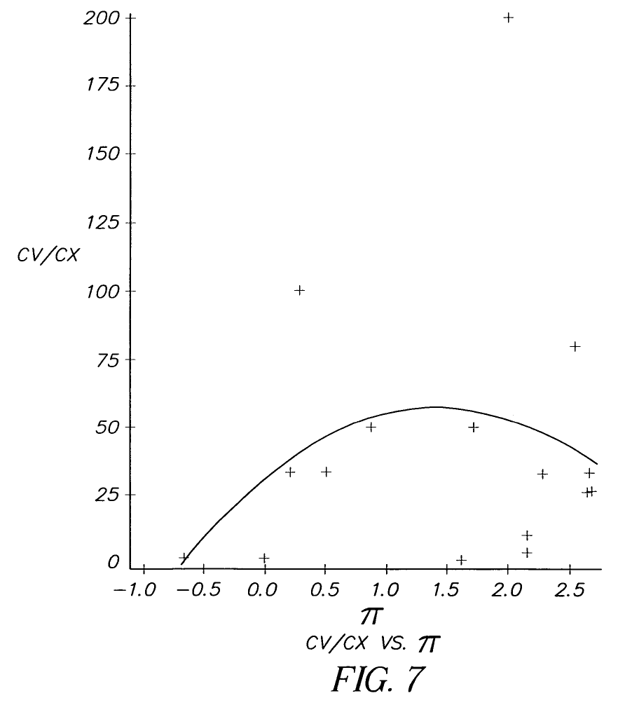

FIG. 6 is a plot of CV/CX versus π. A cubic regression program was used to fit a plot line to the data. For substituents that are too hydrophilic (πis too negative) or too lipophilic (πis too positive), the selectivity of the dye is low. It appears from this data that there is a fairly wide optimum hydrophobicity range (π˜0·4). FIG. 7 is a plot of selectivity versus MR. CV/CX drops very rapidly as the steric parameter increases and then levels off. (For this data set, dyes with the highest selectivity have MR<3.0).
CV/CX vs. π

FIG. 6. Plot of CV/CX versus the 1c parameter for the thiacarbocyanine chromophore.
CV/CX vs. MR

FIG. 7. Plot of CV/CX versus the MR parameter for the thiacarbocyanine chromophore.

As a direct example, consider the N-ethyl, N′-2-hydroxyethyl dye (D-125, selectivity=91) versus the N,N′-di(2-hydroxyethyl) dye, which is not active. The dyes have similar steric parameters (MR=2.29 and 2.52, respectively) but quite different hydrophobicity parameters (0.61 and −1.38). Here the data suggest that hydrophobicity has the largest effect on selectivity.

Also consider the N,N′-di(methylthioethyl) dye (CV/CX=12). This dye has a hydrophobicity parameter in the optimum range (n=1.84) but its steric parameter (MR=4.75) is large, which suggests that it has low selectivity for steric reasons.

Statistical analysis also indicates that both the π and MR parameters are important and it is possible to fit the data to an equation using regression analysis. The equation obtained using this procedure is:
CV/CX=21.8π+0.1 π² −20.6 MR+98.5, (r=0.93, F=6.29, PR>F=0.0826, RMSE=26.0).

As a second example, consider Table XXI, which lists the variation in nitrogen substituents for the benzothiazole-benzoxazole dyes (taken from Table V, 18 examples). Each substituent can again be assigned a hydrophobicity parameter and a steric parameter. A correlation table is also listed (the correlation coefficient between π and MR is 0.38).

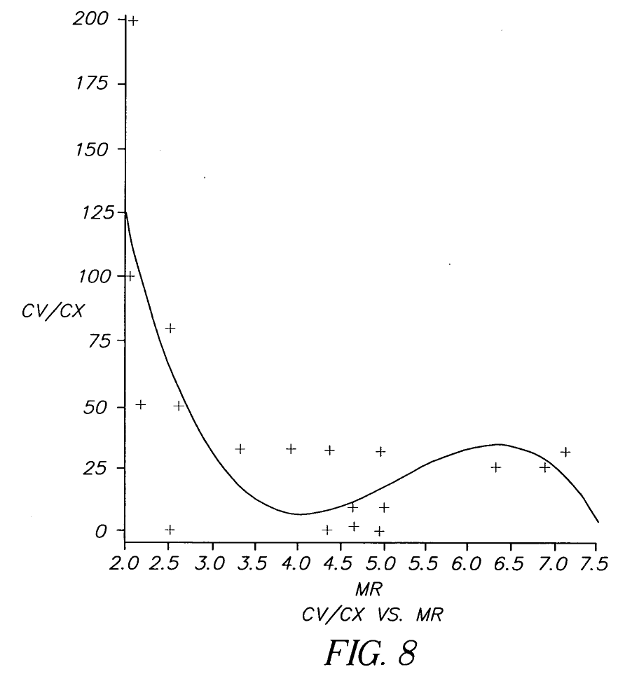

The range of hydrophobicity parameter values is not as large for this data set; however, the same general trend is observed as in the first example, and there appears again to be a fairly broad range of n values in which dyes can have high selectivity (π˜0−>2.5), FIG. 8. Also, FIG. 9 indicates that the dyes' selectivity drops rapidly as the steric parameter increases (dyes with higher selectivity again have MR<3.0).

TABLE XXI
Nitrogen Substituent Parameters for the Oxathiacarbocyanine Chromophore

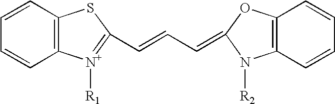

| ID | R1 | R2 | CV | CX | CV/C | π | MR |
|---|---|---|---|---|---|---|---|
| D-124 | CCN | Et | 10.0 | 0.1 | 100.0 | 0.29 | 2.04 |
| D-122 | iPr | Et | 0.4 | <0.005 | >80.0 | 2.55 | 2.53 |
| | CCC═0 | Et | 5.0 | 0.1 | 50.0 | 0.87 | 2.63 |
| | Et | CC(F)3 | 5.0 | 0.1 | 50.0 | 1.73 | 2.18 |
| | CCS02CC | Et | >10.0 | 0.3 | >33.3 | 0.21 | 3.93 |
| | CC(═0)N(Et)2 | Et | >10.0 | 0.3 | >33.3 | 0.51 | 4.40 |
| | CC(═0)Ph-2, 4-OH | Et | >10.0 | 0.3 | >33.3 | 2.27 | 4.99 |
| | CCC═NNS02Ph-4C | Et | 10.0 | 0.3 | 33.3 | | 7.16 |
| | CC(Br)═C | Et | 1.0 | 0.03 | 33.3 | 2.65 | 3.35 |
| | CCNS02Ph-4C | Et | 8.0 | 0.3 | 26.7 | 2.67 | 6.35 |
| | CCC(═O)N(iPr)Ph | Et | 0.8 | 0.03 | 26.7 | 2.64 | 6.91 |
| | CC(═O)N | Et | >10.0 | 1.0 | >10.0 | −0.66 | 2.54 |
| | CC(═0)Ph | Et | 3.0 | 0.3 | 10.0 | 2.15 | 4.68 |
| | Et | C(C═O)Ph | 3.0 | 0.8 | 3.7 | 2.15 | 4.68 |
| | CC(═0)morpholino | Et | >10.0 | >1.0 | 1.0 | −0.01 | 4.37 |
| | CCC═NNC(═S)N | Et | >10.0 | >1.0 | 1.0 | | 5.03 |
| | CC(═0)Ph-3, 4-OH | Et | >10.0 | >1.0 | 1.0 | 1.6 | 4.99 |

Correlation Coefficients

| | | | | |
|---|---|---|---|---|
| CX | CV | CV/CX | π | MR |

| | | | | |
|---|---|---|---|---|
| CV | 1.00000 | −0.37589 | −0.69408 | 0.20050 |
| CV/CX | | 1.00000 | 0.14261 | −0.51577 |
| π | | | 1.00000 | 0.38025 |
| MR | | | | 1.00000 |

CV/CX vs. π

The equation obtained by fitting this data to the two parameters is similar to the previous equation:
CV/CX=33.9 π−6.5 π2−22.7 MR+103.6 (r=0.67, F=3.39 PR>F=0.0538, RMSE=41.5).

As a third example, Table XXII lists representative chain substituents for which well-characterized hydrophobicity (u), steric (MR, L, B1) and electronic (σₘ, σₚ, F, R) parameters are available. In this case, the parameters are very colinear as indicated by the correlation table. For instance, the πand MR values have a correlation coefficient of 0.91.

TABLE XXII
Chain Substituent Parameters

| ID | R | π | MR | F | R | σₘ | σₚ | L | B1 |
|---|---|---|---|---|---|---|---|---|---|
| D- | H | 0.00 | 0.103 | 0.00 | 0.00 | 0.00 | 0.00 | 2.06 | 1.00 |
| 100 | Me | 0.56 | 0.565 | −0.04 | −0.13 | −0.07 | −0.17 | 3.00 | 1.52 |
| | Et | 1.02 | 1.030 | −0.05 | −0.10 | −0.07 | −0.15 | 4.11 | 1.52 |
| | SMe | 0.61 | 1.382 | 0.20 | −0.18 | 0.15 | 0.00 | 4.30 | 1.70 |
| | SEt | 1.07 | 1.842 | 0.23 | −0.18 | 0.18 | 0.03 | 5.24 | 1.70 |
| | 2- | 1.61 | 2.404 | 0.10 | 0.04 | 0.09 | 0.05 | 5.97 | 1.65 |
| | Thienyl. | | | | | | | | |

Correlation Coefficients

| | π | MR | F | R | σₘ | σₚ | | B1 |
|---|---|---|---|---|---|---|---|---|
| π | 1.000 | 0.912 | 0.293 | 0.132 | 0.303 | 0.226 | 0.937 | 0.735 |
| MR | | 1.000 | 0.640 | 0.028 | 0.657 | 0.545 | 0.992 | 0.786 |
| F | | | 1.000 | −0.403 | 0.990 | 0.753 | 0.603 | 0.570 |
| R | | | | 1.000 | −0.277 | 0.265 | −0.025 | −0.507 |
| aₘ | | | | | 1.000 | 0.837 | 0.609 | 0.500 |
| a | | | | | | 1.000 | 0.465 | 0.084 |
| L^{p} | | | | | | | 1.000 | 0.815 |
| B | | | | | | | | 1.000 |

One could apply this technique in a similar fashion to the other tables, such as variations in back-ring substituents, incorporating other parameters where appropriate. However, in the present case, the major problem with this approach is the limited number of compounds in which only one substituent in one position is varied and the fact that some of the IC₅₀ values are uncertain (e.g., CX <0.005 for many dyes).

The invention has been described in detail with particular reference to certain preferred embodiments thereof, but it will be understood that variations and modifications can be effected within the spirit and scope of the invention.

1. A method for selecting pharmacological compounds for selective inhibition of cancer cells comprising:
determining the reduction potential (E_{R}) of said compound; and
selecting said compound which has a reduction potential from −1.1 to −0.8 volts.

2. The method of claim 1 wherein said selective inhibition of cancer cells to non-cancer cells is at least 60:1.

3. The method of claim 1 wherein said compound is a methine dye.

4. The method of claim 3 wherein said methine dye is a cyanine dye, merocyanine dye or oxonol dye.

5. The method of claim 4 wherein the pharmacological compound is represented by Formula (1a) 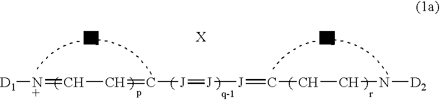
wherein:
E₁ and E₂ independently represent the atoms necessary to form a substituted or unsubstituted heterocyclic basic nucleus;
each J independently represents a substituted or unsubstituted methine group;
each q is a positive integer of from 1 to 4;
each p is 0 or 1;
each r is 0 or 1;
D₁ and D₂ each independently represent a substituted or unsubstituted alkyl group or a substituted or unsubstituted aryl group, provided at least one of D₁ and D₂ includes a cationic group; and
X represents one or more pharmaceutically acceptable anions.

6. The method of claim 5 wherein q is 2.

7. The method of claim 5 wherein each J independently represents an unsubstituted methine group.

8. The method of claim 5 wherein E₁ and E₂ independently represent the atoms necessary to complete a substituted or unsubstituted benzothiazole, benzoxazole or quinoline nucleus.

9. The method of claim 5 wherein at least one of D₁ and D₂ includes a tetravalent nitrogen atom.

10. The method of claim 9 wherein said tetravalent nitrogen is a trialkylammonium salt having substituted or unsubstituted alkyl groups.

11. The method of claim 9 wherein said tetravalent nitrogen is in an aromatic ring having a positive delocalized charge.

12. The method of claim 5 wherein at least one of D₁ and D₂ is selected from the group consisting of:

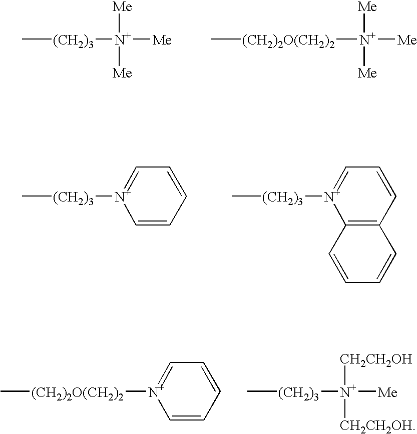

13. The method of claim 5 wherein said pharmaceutically acceptable anions include chloride, acetate, propionate, valerate, citrate, maleate, fumarate, lactate, succinate, tartrate and benzoate.

14. The method of claim 4 wherein the dye is represented by Formula (1b), 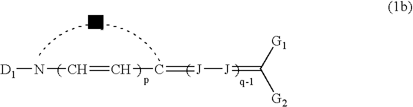
wherein:
E₁ represents the atoms necessary to form a substituted or unsubstituted heterocyclic basic nucleus;
each J independently represents a substituted or unsubstituted methine group;
each q is a positive integer of from 1 to 4;
each p is 0 or 1;
G₁ and G₂ each independently represent an electron-accepting group; and
D₁ represents a substituted alkyl group or a substituted aryl group, wherein D₁ includes a cationic substituent.

15. The method of claim 14 wherein q is 2.

16. The method of claim 14 wherein each J independently represents an unsubstituted methine group.

17. The method of claim 14 wherein E₁ represents the atoms necessary to complete a substituted or unsubstituted benzothiazole, benzoxazole or quinoline nucleus.

18. The method of claim 14 wherein G₁ and G₂ combine together to form a ring that comprises an acidic nucleus.

19. The method of claim 14 wherein at least one of D₁ and D₂ includes a tetravalent nitrogen atom.

20. The method of claim 19 wherein said tetravalent nitrogen is a trialkylammonium salt having substituted or unsubstituted alkyl groups.

21. The method of claim 19 wherein said tetravalent nitrogen is in an aromatic ring having a positive delocalized charge.

22. The method of claim 14 wherein at least one of D₁ and D₂ is selected from the group consisting of:

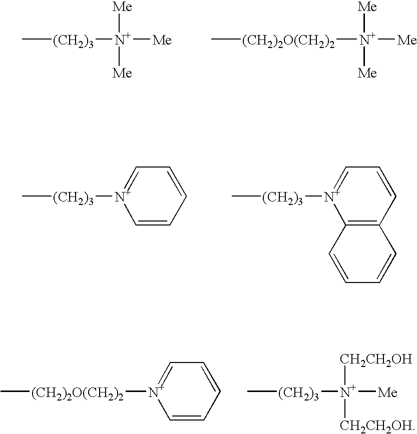

23. The method of claim 4 wherein the dye is represented by Formula (1c), 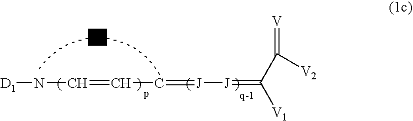
wherein:
E₁ represents the atoms necessary to form a substituted or unsubstituted heterocyclic basic nucleus;
each J independently represents a substituted or unsubstituted methine group;
each q is a positive integer of from 1 to 4;
each p is 0 or 1;
V represents an electron-accepting group;
V₁ represents an electron-withdrawing group;
V₂ represents a substituted or unsubstituted aromatic group or substituted or unsubstituted alkyl group; and
D₁ represents a substituted alkyl group or a substituted aryl group, wherein D₁ includes a cationic substituent.

24. The method of claim 23 wherein q is 2.

25. The method of claim 1 further comprising first identifying a compound containing at least one cationic substituent;

26. The method of claim 25 wherein said at least one cationic substituent includes a tetravalent nitrogen atom.

27. The method of claim 25 wherein at least one cationic substituent includes a substituted or unsubstituted tetralkylammonium group.

28. The method of claim 25 wherein at least one substituent includes a substituted or unsubstituted cationic aromatic group.

29. The method of claim 25 wherein at least one cationic substituent includes a substituted or unsubstituted pyridinium group.

30. The method of claim 25 wherein at least one cationic substituent is represented by Formula (C-2), 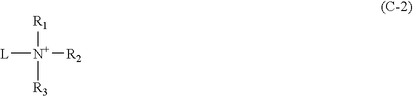
wherein:
L represents a linking group; and
R₁, R₂, and R₃ independently represent substituted or unsubstituted alkyl, or substituted or unsubstituted aryl groups, provided that two of R₁, R₂, and R₃ are capable of joining together to form a ring.

31. The pharmacological compound of claim 25 wherein at least one cationic substituent is represented by Formula (C-3), 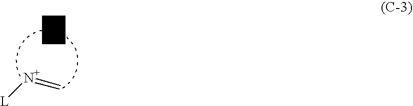
wherein:
L represents a linking group; and
Ar represents the atoms necessary to complete a substituted or unsubstituted aromatic ring.

32. The method of claim 4 wherein said cyanine dye is a carbocyanine having an unsubstituted 3 methine carbon chain.

33. The method of claim 4 wherein said cyanine dye is a selenathiacarbocyanine dye having back-ring substitution.

34. The method of claim 3 wherein the molar refractivity value, MR, of said methine dye is less than 3.0.

35. The method of claim 1 further comprising selecting a compound having high aqueous solubility.

36. The method of claim 1 further comprising enhancing the selective inhibition of cancer cells of said compound by adding a hydrophilic substituent to increase aqueous solubility.

37. A pharmacological compound comprising at least one cyanine dye or merocyanine dye, wherein the dye has at least one cationic substituent, wherein the dye has a reduction potential of from −1.1 to 0.8 volts, and wherein said pharmacological compound demonstrates selective inhibition of cancer cells.

38. The pharmacological compound of claim 37 wherein said selective inhibition of cancer cells to non-cancer cells is at least 60:1.

39. The pharmacological compound of claim 37 wherein at least one cationic substituent includes a tetravalent nitrogen atom.

40. The pharmacological compound of claim 37 wherein at least one cationic substituent includes a substituted or unsubstituted tetralkylammonium group.

41. The pharmacological compound of claim 37 wherein at least one substituent includes a substituted or unsubstituted cationic aromatic group.

42. The pharmacological compound of claim 37 wherein at least one cationic substituent includes a substituted or unsubstituted pyridinium group.

43. The pharmacological compound of claim 37 wherein at least one cationic substituent is represented by Formula (C-2), 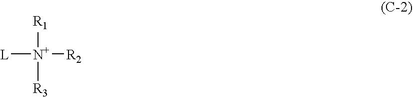
wherein:
L represents a linking group; and
R₁, R₂, and R₃ independently represent substituted or unsubstituted alkyl, or substituted or unsubstituted aryl groups, provided that two of R₁, R₂, and R₃ are capable of joining together to form a ring.

44. The pharmacological compound of claim 37 wherein at least one cationic substituent is represented by Formula (C-3), 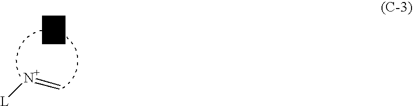
wherein:
L represents a linking group; and
Ar represents the atoms necessary to complete a substituted or unsubstituted aromatic ring.

45. The pharmacological compound of claim 37 wherein the pharmacological compound is represented by Formula (1a) 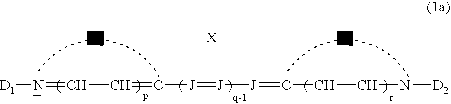
wherein:
E₁ and E₂ independently represent the atoms necessary to form a substituted or unsubstituted heterocyclic basic nucleus;
each J independently represents a substituted or unsubstituted methine group;
each q is a positive integer of from 1 to 4;
each p is 0 or 1;
each r is 0 or 1; and
D₁ and D₂ each independently represent a substituted or unsubstituted alkyl group or a substituted or unsubstituted aryl group, provided at least one of D₁ and D₂ includes a cationic group.

46. The pharmacological compound of claim 45 wherein q is 2.

47. The pharmacological compound of claim 45 wherein each J independently represents an unsubstituted methine group.

48. The pharmacological compound of claim 45 wherein E₁ and E₂ independently represent the atoms necessary to complete a substituted or unsubstituted benzothiazole, benzoxazole or quinoline nucleus.

49. The pharmacological compound of claim 37 wherein the dye is represented by Formula (1b), 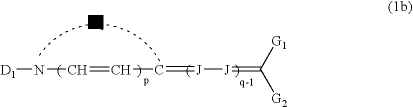
wherein:
E₁ represents the atoms necessary to form a substituted or unsubstituted heterocyclic basic nucleus;
each J independently represents a substituted or unsubstituted methine group;
each q is a positive integer of from 1 to 4;
each p is 0 or 1;
G₁ and G₂ each independently represent an electron-accepting group; and
D₁ represents a substituted alkyl group or a substituted aryl group, wherein D₁ includes a cationic substituent.

50. The pharmacological compound of claim 49 wherein q is 2.

51. The pharmacological compound of claim 49 wherein each J independently represents an unsubstituted methine group.

52. The pharmacological compound of claim 49 wherein E₁ represents the atoms necessary to complete a substituted or unsubstituted benzothiazole, benzoxazole or quinoline nucleus.

53. The pharmacological compound of claim 49 wherein G₁ and G₂ combine together to form a ring that comprises an acidic nucleus.

54. The pharmacological compound of claim 37 wherein the dye is represented by Formula (1c), 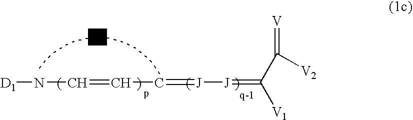
wherein:
E₁ represents the atoms necessary to form a substituted or unsubstituted heterocyclic basic nucleus;
each J independently represents a substituted or unsubstituted methine group;
each q is a positive integer of from 1 to 4;
each p is 0 or 1;
V represents an electron-accepting group;
V₁ represents an electron-withdrawing group;
V₂ represents a substituted or unsubstituted aromatic group or substituted or unsubstituted alkyl group; and
D₁ represents a substituted alkyl group or a substituted aryl group, wherein D₁ includes a cationic substituent.

55. The pharmacological compound of claim 54 wherein q is 2.
